(19) United States
(12) Patent Application Publication
(10) Pub. No.: US 2023/0149377 A1
(43) Pub. Date: May 18, 2023

(54) PHARMACEUTICAL METHODS

(71) Applicant: ELEVAR THERAPEUTICS, INC., Salt Lake City, UT (US)

(72) Inventors: Arlo N. MCGINN, Salt Lake City, UT (US); Bumjin KIM, Midvale, UT (US); Sung Chul KIM, North Salt Lake, UT (US); Cheol Hee PARK, Holladay, UT (US)

(21) Appl. No.: 17/295,848

(22) PCT Filed: Nov. 20, 2019
(86) PCT No.: PCT/US2019/062494

§ 371 Date: May 20, 2021

Related U.S. Application Data
(60) Provisional application No. 62/770,437, filed on Nov. 21, 2018.

(51) Int. Cl. A61K 31/444; A61K 31/337; A61K 31/573; A61K 31/138; A61K 31/426; A61K 45/06; A61P 35/00; A61P 1/00

(52) U.S. Cl. CPC A61K 31/444; A61K 31/337; A61K 31/573; A61K 31/138; A61K 31/426; A61K 45/06; A61P 35/00; A61P 1/00

(57) ABSTRACT

Provided herein are methods for the treatment of diseases, comprising administering a combination of a tyrosine kinase inhibitors and tubulin binding agent.

### CROSS-REFERENCE

This application claims the benefit of U.S. Provisional Application No. 62/770,437, filed on Nov. 21, 2018, which is incorporated herein by reference in its entirety.

### BACKGROUND OF THE DISCLOSURE

Cancer remains one of the most deadly threats to human health, affecting over 1 million new patients each year in the United States. Although there have been significant advances in the medical treatment of certain cancers, current methods of treatment remain relatively non-selective: surgery removes the diseased tissue; radiotherapy shrinks solid tumors; and chemotherapy kills rapidly dividing cells. These treatments may result in numerous side effects, in some cases so severe as to limit the dosage that can be given and thus preclude the use of potentially effective drugs.

Gastric cancer is the fourth most common cancer world-wide and the fourth most common cause of cancer-related death. Surgical resection is effective for complete cure of localized cancer. However, patients with locally advanced, metastatic, or recurrent cancer are inoperable. For such patients, extending the duration of survival by systemic chemotherapy is the primary treatment; yet, the response rate for first-line therapy in patients with advanced gastric cancer is below 50%. Most patients who respond to first-line chemotherapy, still experience tumor progression, often developing peritoneal metastases and exacerbation of systemic conditions, resulting in 20-50% of patients requiring second-line chemotherapy.

Rivoceranib (also known as YN968D1, developed in China as apatinib and marketed as Aitan®) is an orally administered small molecule tyrosine kinase inhibitor with selectivity towards the VEGFR-2/kinase insert domain receptor. Rivoceranib has received approval in China, for treatment of advanced gastric cancer, and has received orphan medicinal product designation for the treatment of gastric cancer from Europe, the FDA, and the MFDS in South Korea. According to a recent review (see L. J. Scott, “Apatinib: A Review in Advanced Gastric Cancer and Other Advanced Cancers,” Drugs, 2018, 78(7), 747-758), “further clinical experience and long-term pharmacovigilance data are required to more definitively establish the efficacy and safety profile of apatinib, including its use in combination with other chemotherapy agents . . . ”

Paclitaxel is a tubulin binding agent, approved for the treatment of ovarian cancer, breast cancer, Kaposi's sarcoma and lung cancer. It is administered by intravenous injection.

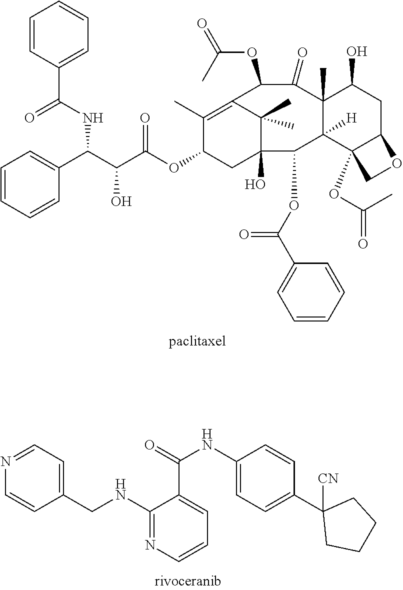

A recent article (see Wong et al, “Estimation of clinical trial success rates and related parameters”, Biostatistics, 2018) found less than 14% of clinical trials eventually lead to an approved drug, while clinical trials in the oncology field have the lowest success rate of only 3.4%.

There exists a need for an effective treatment of cancer and neoplastic diseases.

There exists a need for effective treatment of cancer with reduced treatment toxicity.

There exists a need for effective treatment of cancer with fewer side effects.

There exists a need for effective treatment of cancer with less severe side effects.

There exists a need for effective treatment of cancer allowing for administration of lower doses.

There exists a need for effective treatment of cancer with longer survival rates.

There exists a need for effective treatment of cancer with more convenient administration methods.

There exists a need for combination therapy modalities to treat cancer and other diseases.

### SUMMARY OF THE DISCLOSURE

Provided herein are methods for treating diseases. Further provided herein are methods for treating diseases, comprising administering rivoceranib, or a pharmaceutically acceptable salt thereof and paclitaxel, or a pharmaceutically acceptable salt thereof. Further provided herein are methods for treating cancer, comprising administering rivoceranib, or a pharmaceutically acceptable salt thereof and paclitaxel, or a pharmaceutically acceptable salt thereof. Further provided herein are methods for treating cancer, comprising administering rivoceranib and paclitaxel, or a pharmaceutically acceptable salt thereof. Further provided herein are methods for treating cancer, comprising administering, a pharmaceutically acceptable salt of rivoceranib and paclitaxel, or a pharmaceutically acceptable salt thereof. Further provided herein are methods for treating cancer, comprising administering, rivoceranib mesylate and paclitaxel, or a pharmaceutically acceptable salt thereof.

Further provided herein are methods for treating cancer, wherein the rivoceranib is administered in an amount of from 100 mg to 1000 mg. Further provided herein are methods for treating cancer, wherein the rivoceranib is administered in an amount of from 150 mg to 800 mg. Further provided herein are methods for treating cancer, wherein the rivoceranib is administered in an amount of from 200 mg to 700 mg. Further provided herein are methods for treating cancer, wherein the rivoceranib is administered in an amount of less than 700 mg. Further provided herein are methods for treating cancer, wherein the rivoceranib is administered in an amount of about 200 mg. Further provided herein are methods for treating cancer, wherein the rivoceranib is administered in an amount of about 300 mg. Further provided herein are methods for treating cancer, wherein the rivoceranib is administered in an amount of about 400 mg. Further provided herein are methods for treating cancer, wherein the rivoceranib is administered in an amount of about 500 mg. Further provided herein are methods for treating cancer, wherein the rivoceranib is administered in an amount of about 600 mg. Further provided herein are methods for treating cancer, wherein the total daily dose of the rivoceranib is less than 700 mg. Further provided herein are methods for treating cancer, wherein the total daily dose of the rivoceranib is less than 685 mg. Further provided herein are methods for treating cancer, wherein the rivoceranib is administered orally. Further provided herein are methods for treating cancer, wherein the rivoceranib is administered as a dried powder, a liquid, a capsule, a pellet or a tablet. Further provided herein are methods for treating cancer, wherein the rivoceranib is administered as a tablet. Further provided herein are methods for treating cancer, wherein the tablet is a film coated tablet. Further provided herein are methods for treating cancer, wherein the tablet comprises the rivoceranib in an amount of about 100 mg. Further provided herein are methods for treating cancer, wherein the tablet comprises the rivoceranib in an amount of about 200 mg. Further provided herein are methods for treating cancer, wherein the tablet further comprises one or more of pregelatinized starch, microcrystalline cellulose, sodium starch glycolate, povidone (K-29/32), colloidal silicon dioxide, magnesium stearate and Opadry white. Further provided herein are methods for treating cancer, wherein the rivoceranib is administered once daily. Further provided herein are methods for treating cancer, wherein the rivoceranib is administered twice daily.

Further provided herein are methods for treating diseases, comprising administering rivoceranib, or a pharmaceutically acceptable salt thereof and paclitaxel. Further provided herein are methods for treating diseases, comprising administering rivoceranib, or a pharmaceutically acceptable salt thereof and a pharmaceutically acceptable salt of paclitaxel. Further provided herein are methods for treating cancer, comprising administering paclitaxel in an amount of no more than 100 mg/m². Further provided herein are methods for treating cancer, wherein the paclitaxel is administered in an amount of 10 mg/m² to 100 mg/m². Further provided herein are methods for treating cancer, wherein the paclitaxel is administered in an amount of 50 mg/m² to 90 mg/m². Further provided herein are methods for treating cancer, wherein the paclitaxel is administered in an amount of about 80 mg/m². Further provided herein are methods for treating cancer, wherein the paclitaxel is administered orally or parenterally. Further provided herein are methods for treating cancer, wherein the paclitaxel is administered parenterally. Further provided herein are methods for treating cancer, wherein the parenteral administration is selected from intravenous, intradermal, intramuscular or subcutaneous administration. Further provided herein are methods for treating cancer, wherein the paclitaxel is administered intravenously. Further provided herein are methods for treating cancer, wherein the paclitaxel is administered after the administration of the rivoceranib. Further provided herein are methods for treating cancer, wherein the paclitaxel is administered about an hour after the administration of the rivoceranib. Further provided herein are methods for treating cancer, wherein the paclitaxel is administered over a period of less than one hour. Further provided herein are methods for treating cancer, wherein the paclitaxel is administered over a period of about an hour. Further provided herein are methods for treating cancer, wherein the paclitaxel is administered over a period of 30-60 minutes. Further provided herein are methods for treating cancer, wherein the paclitaxel is administered no more than once a week. Further provided herein are methods for treating cancer, wherein the paclitaxel is administered at least once a week. Further provided herein are methods for treating cancer, wherein the paclitaxel is administered once a week. Further provided herein are methods for treating cancer, wherein the paclitaxel is administered once a month. Further provided herein are methods for treating cancer, wherein the paclitaxel is administered twice a month. Further provided herein are methods for treating cancer, wherein the paclitaxel is administered three times a month. Further provided herein are methods for treating cancer, wherein the paclitaxel is administered as a composition comprising nanoparticles comprising paclitaxel and a protein carrier. Further provided herein are methods for treating cancer, wherein the paclitaxel is administered as a composition comprising nanoparticles comprising paclitaxel and albumin. Further provided herein are methods for treating cancer, wherein the paclitaxel in the nanoparticles is coated with the albumin. Further provided herein are methods for treating cancer, wherein the nanoparticles have an average diameter of no greater than about 200 nm. Further provided herein are methods for treating cancer, wherein the nanoparticles have an average diameter of no greater than about 200 nm. Further provided herein are methods for treating cancer, wherein the weight ratio of albumin and paclitaxel in the nanoparticle composition is about 9:1. Further provided herein are methods for treating cancer, wherein prior to the administration of the paclitaxel, premedication is administered. Further provided herein are methods for treating cancer, wherein the premedication comprises one or more of dexamethasone, diphenhydramine or famotidine. Further provided herein are methods for treating cancer, wherein the premedication comprises dexamethasone, diphenhydramine and famotidine. Further provided herein are methods for treating cancer, wherein the premedication comprises 20 mg dexamethasone, 50 mg diphenhydramine and 20 mg famotidine. Further provided herein are methods for treating cancer, wherein the premedication is administered intravenously. Further provided herein are methods for treating cancer, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof. Further provided herein are methods for treating cancer, wherein the cancer is a cancer that affects the digestive system. Further provided herein are methods for treating cancer, wherein the cancer is anal cancer, bowel cancer, colon cancer, colorectal cancer, esophageal cancer, gallbladder and biliary tract cancer, gastric cancer, gastro-intestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof. Further provided herein are methods for treating cancer, wherein the cancer is gastric cancer. Further provided herein are methods for treating cancer, wherein the cancer is gastroesophageal junction cancer. Further provided herein are methods for treating cancer, wherein the cancer is advanced gastric or gastroesophageal junction cancer. Further provided herein are methods for treating cancer, wherein the cancer is recurrent metastatic gastric or gastroesophageal junction cancer. Further provided herein are methods for treating cancer, wherein the cancer is metastatic gastric or gastroesophageal junction cancer. Further provided herein are methods for treating cancer, wherein the method is a second line therapy.

Further provided herein are methods for treating cancer, further comprising administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, β2-agonists, anticholinergic agents, hormonal agents, biological agents, tubulin binding agents, glucocorticoids, corticosteroid agents, antibacterial agents, antihistamines, antiemetic agents, anti-malarial agents, anti-viral agents, and antibiotics; and, optionally with radiation therapy.

Further provided herein are methods for treating cancer, further comprising administering radiation therapy. Further provided herein are methods for treating cancer, wherein the cancer comprises a lesion. Further provided herein are methods for treating cancer, wherein the lesion is measured before administration and either in between a plurality of administrations or after the administration or both. Further provided herein are methods for treating cancer, wherein the lesion is measured by radiological assessments using computerized tomography scan or magnetic resonance imaging. Further provided herein are methods for treating cancer, wherein the lesion has reduced in size after the administration of the combination. Further provided herein are methods for treating cancer, wherein the lesion has reduced in size by at least 10%. Further provided herein are methods for treating cancer, wherein the lesion has reduced in size by at least 20%. Further provided herein are methods for treating cancer, wherein the lesion has reduced in size by at least 50%. Further provided herein are methods for treating cancer, wherein the lesion has reduced in size by at least 75%.

Further provided herein are methods for treating cancer, comprising administering a combination of a tyrosine kinase inhibitor, or a pharmaceutically acceptable salt thereof; and a tubulin binding agent, or a pharmaceutically acceptable salt thereof. Further provided herein are methods for treating cancer, wherein the tyrosine kinase inhibitor is a vascular endothelial growth factor receptor (VEGF) inhibitor. Further provided herein are methods for treating cancer, wherein the tyrosine kinase inhibitor is a selective vascular endothelial growth factor receptor-2 (VEGF2) inhibitor. Further provided herein are methods for treating cancer, wherein the tyrosine kinase inhibitor is afatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, ranibizumab, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib.

Further provided herein are methods for treating cancer, wherein the tyrosine kinase inhibitor is rivoceranib. Further provided herein are methods for treating cancer, wherein the tyrosine kinase inhibitor is rivoceranib mesylate. Further provided herein are methods for treating cancer, wherein the tyrosine kinase inhibitor is administered in an amount of from 150 mg to 800 mg. Further provided herein are methods for treating cancer, wherein the tyrosine kinase inhibitor is administered in an amount of from 200 mg to 700 mg. Further provided herein are methods for treating cancer, wherein the tyrosine kinase inhibitor is administered in an amount of less than 700 mg. Further provided herein are methods for treating cancer wherein the tyrosine kinase inhibitor is administered in an amount of about 200 mg. Further provided herein are methods for treating cancer, wherein the tyrosine kinase inhibitor is administered in an amount of about 300 mg. Further provided herein are methods for treating cancer, wherein the tyrosine kinase inhibitor is administered in an amount of about 400 mg. Further provided herein are methods for treating cancer, wherein the tyrosine kinase inhibitor is administered in an amount of about 500 mg. Further provided herein are methods for treating cancer, wherein the tyrosine kinase inhibitor is administered in an amount of about 600 mg. Further provided herein are methods for treating cancer, wherein the total daily dose of the tyrosine kinase inhibitor is less than 700 mg. Further provided herein are methods for treating cancer, wherein the total daily dose of the tyrosine kinase inhibitor is less than 685 mg. Further provided herein are methods for treating cancer, wherein the tyrosine kinase inhibitor is administered orally.

Further provided herein are methods for treating cancer, wherein the tyrosine kinase inhibitor is administered as a tablet. Further provided herein are methods for treating cancer, wherein the tyrosine kinase inhibitor is administered once daily. Further provided herein are methods for treating cancer, wherein the tyrosine kinase inhibitor is administered twice daily.

Further provided herein are methods for treating cancer, wherein the tubulin binding agent is a taxane. Further provided herein are methods for treating cancer, wherein the taxane is paclitaxel, Docetaxel, Cabazitaxel, Larotaxel, Orataxel, Tesetaxel, Milataxel, Taxoprexin, docetaxel-d₆-t-Boc, docetaxel-f3-t-Boc, cabazitaxel-7,10-d₆, abeo-taxane15a.2, BMS-184476, BMS-188797, BMS-275183, SB-T-1214, SB-T-1216, SB-T-12854, SB-T-121602, SB-CST-10202 or DHA-SB-T-1214. Further provided herein are methods for treating cancer, wherein the tubulin binding agent is administered in an amount of no more than 100 mg/m². Further provided herein are methods for treating cancer, wherein the tubulin binding agent is administered in an amount of 10 mg/m² to 100 mg/m². Further provided herein are methods for treating cancer, wherein the tubulin binding agent is administered in an amount of 50 mg/m² to 90 mg/m². Further provided herein are methods for treating cancer, wherein the tubulin binding agent is administered in an amount of about 80 mg/m². Further provided herein are methods for treating cancer, wherein the tubulin binding agent is administered orally or parenterally. Further provided herein are methods for treating cancer, wherein the tubulin binding agent is administered parenterally. Further provided herein are methods for treating cancer, wherein the tubulin binding agent is administered intravenously. Further provided herein are methods for treating cancer, wherein the tubulin binding agent is administered after the administration of the tyrosine kinase inhibitor. Further provided herein are methods for treating cancer, wherein the tubulin binding agent is administered about an hour after the administration of the tyrosine kinase inhibitor. Further provided herein are methods for treating cancer, wherein the tubulin binding agent is administered over a period of less than one hour. Further provided herein are methods for treating cancer, wherein the tubulin binding agent is administered over a period of about an hour. Further provided herein are methods for treating cancer, wherein the tubulin binding agent is administered over a period of 30-60 minutes. Further provided herein are methods for treating cancer, wherein the tubulin binding agent is administered no more than once a week. Further provided herein are methods for treating cancer, wherein the tubulin binding agent is administered at least once a week. Further provided herein are methods for treating cancer, wherein the tubulin binding agent is administered once a week. Further provided herein are methods for treating cancer, wherein the tubulin binding agent is administered once a month. Further provided herein are methods for treating cancer, wherein the tubulin binding agent is administered twice a month. Further provided herein are methods for treating cancer, wherein prior to the administration of the tubulin binding agent, premedication is administered. Further provided herein are methods for treating cancer, wherein the premedication comprises one or more of dexamethasone, diphenhydramine or famotidine. Further provided herein are methods for treating cancer, wherein the premedication comprises dexamethasone, diphenhydramine and famotidine. Further provided herein are methods for treating cancer, wherein the premedication comprises 20 mg dexamethasone, 50 mg diphenhydramine and 20 mg famotidine. Further provided herein are methods for treating cancer, wherein the premedication is administered intravenously. Further provided herein are methods for treating cancer, wherein the tubulin binding agent is administered.

Further provided herein are methods for treating cancer, wherein the rivoceranib and the paclitaxel act synergistically to treat the cancer. Further provided herein are methods for treating cancer, wherein the administration of the rivoceranib and the paclitaxel is more effective than administering the rivoceranib alone or paclitaxel alone. Further provided herein are methods for treating cancer, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof. Further provided herein are methods for treating cancer, wherein the cancer is a cancer that affects the digestive system. Further provided herein are methods for treating cancer, wherein the cancer is anal cancer, bowel cancer, colon cancer, colorectal cancer, esophageal cancer, gallbladder and biliary tract cancer, gastric cancer, gastro-intestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof. Further provided herein are methods for treating cancer, wherein the cancer is gastric cancer. Further provided herein are methods for treating cancer, wherein the cancer is gastroesophageal junction cancer. Further provided herein are methods for treating cancer, wherein the cancer is advanced gastric or gastroesophageal junction cancer. Further provided herein are methods for treating cancer, wherein the cancer is recurrent metastatic gastric or gastroesophageal junction cancer. Further provided herein are methods for treating cancer, wherein the cancer is metastatic gastric or gastroesophageal junction cancer. Further provided herein are methods for treating cancer, wherein the method is a second line therapy. Further provided herein are methods for treating cancer, further comprising administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, .beta.2-agonists, anticholinergic agents, hormonal agents, biological agents, tubulin binding agents, glucocorticoids, corticosteroid agents, antibacterial agents, antihistamines, anti-malarial agents, anti-viral agents, and antibiotics; and, optionally with radiation therapy. Further provided herein are methods for treating cancer, wherein the cancer comprises a lesion. Further provided herein are methods for treating cancer, wherein the lesion is measured before administering the combination and either in between a plurality of the administrations of the combination or after the administration of the combination or both. Further provided herein are methods for treating cancer, wherein the lesion is measured by radiological assessments using computerized tomography scan or magnetic resonance imaging. Further provided herein are methods for treating cancer, wherein the lesion has reduced in size after the administration of the combination. Further provided herein are methods for treating cancer, wherein the lesion has reduced in size by at least 10%. Further provided herein are methods for treating cancer, wherein the lesion has reduced in size by at least 20%. Further provided herein are methods for treating cancer, wherein the lesion has reduced in size by at least 50%. Further provided herein are methods for treating cancer, wherein the lesion has reduced in size by at least 75%. Further provided herein are methods for treating cancer, wherein the lesion has reduced in size by more than if rivoceranib alone or paclitaxel alone had been administered.

Further provided herein are methods for treating cancer, comprising administering a combination of rivoceranib, or a pharmaceutically acceptable salt thereof, and no more than 80 mg/m² paclitaxel.

Further provided herein are methods for treating cancer, comprising administering a combination of no more than 685 mg rivoceranib, or a pharmaceutically acceptable salt thereof and paclitaxel.

Further provided herein are methods for treating cancer, comprising administering a combination of no more than 685 mg rivoceranib, or a pharmaceutically acceptable salt thereof and no more than 80 mg/m² paclitaxel.

Further provided herein are methods for treating cancer, comprising administering a combination of rivoceranib, or a pharmaceutically acceptable salt thereof and paclitaxel, wherein the rivoceranib and the paclitaxel act synergistically.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 represents the study scheme for the clinical study described in Example 1, to evaluate the safety, tolerability, and efficacy of rivoceranib in combination with paclitaxel in advanced gastric or gastroesophageal junction cancer.

### DETAILED DESCRIPTION OF THE DISCLOSURE

Described herein are methods for treating proliferative diseases, in particular, methods for treating cancer. The methods comprise administering a combination of a tyrosine kinase inhibitor, or a pharmaceutically acceptable salt thereof, and a tubulin binding agent, or a pharmaceutically acceptable salt thereof. Also described, are methods for enhancing the efficacy of a tyrosine kinase inhibitor to treat cancer, comprising administering the tyrosine kinase inhibitor in combination with a tubulin binding agent. Also described, are methods for enhancing the efficacy of a tubulin binding agent to treat cancer, comprising administering the tubulin binding agent in combination with a tyrosine kinase inhibitor. The combination of the tyrosine kinase inhibitor, or a pharmaceutically acceptable salt thereof with the tubulin binding agent, or a pharmaceutically acceptable salt thereof, enhances the efficacy of either of the agents alone, to treat cancer. In some embodiments, the combination of the tyrosine kinase inhibitor, or a pharmaceutically acceptable salt thereof with the tubulin binding agent, or a pharmaceutically acceptable salt thereof, act synergistically to treat cancer. Also described, are methods for inhibiting a cancer associated tumor growth comprising administering a combination of a tyrosine kinase inhibitor, or a pharmaceutically acceptable salt thereof, and a tubulin binding agent, or a pharmaceutically acceptable salt thereof.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

The present invention is generally directed to therapies that are useful to alleviate, abate or eliminate one or more diseases or conditions in a subject in need thereof, as further described herein. In particular, described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof.

### Definitions

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The singular forms “a,” “an,” and, “the” include plural referents unless the context clearly dictates otherwise. Thus, for example, reference to “the surfactant” includes reference to one or more specific surfactants, reference to “an antioxidant” includes reference to one or more of such additives.

The term “subject” as used herein refers to a mammal (e.g., a human, mouse, rat, guinea pig, dog, cat, horse, cow, pig, or non-human primate, such as a monkey, chimpanzee or baboon).

“Effective amount,” and “sufficient amount” may be used interchangeably, and refer to an amount of a substance that is sufficient to achieve an intended purpose or objective.

A “therapeutically effective amount” when used in connection with a pharmaceutical composition described herein is an amount of one or more pharmaceutically active agent(s) sufficient to produce a therapeutic result in a subject in need thereof.

“Therapeutically equivalent” when used in connection with a pharmaceutical composition described herein refers to an amount or quantity of a pharmaceutically acceptable salt of a pharmaceutically active agent that is equivalent to the therapeutically effective amount of the free base of the pharmaceutically active agent.

### Tyrosine Kinase Inhibitors

Various embodiments described herein are directed to methods for treating diseases, comprising administering a tyrosine kinase inhibitor, or a pharmaceutically acceptable thereof.

Tyrosine kinases (or protein tyrosine kinases, PTK) are enzymes that activate and regulate cell proliferation signaling pathways. Overexpression of the PTK gene enhances PTK activity, altering its downstream signaling pathways, causing cell proliferation disorders, and eventually leading to tumor formation. Protein tyrosine kinases occur in two forms—Receptor PTK (RTK) and Non-receptor PTK (NRTK). Receptor RTKs include the epidermal growth factor receptor (EGFR), platelet-derived growth factor receptor (PDGFR), vascular endothelial growth factor receptor (VEGFR) and insulin receptor (InsR) families. They comprise an extracellular binding domain, a transmembrane region, and an intracellular kinase domain that selectively binds to and phosphorylates the substrate. RTK can bind to ligands and phosphorylate tyrosine residues of target proteins and transmit information to activate a series of biochemical reactions; or different information combined to cause a comprehensive cellular response (such as cell proliferation). Clinical studies in cancer have shown that these receptors and their ligands play a significant role in tumor formation and/or growth. Many cancers have over-expressed growth factors that cause excessive tyrosine phosphorylation signal into cells.

VEGFR family members include VEGFR1, VEGFR2 and VEGFR3. The family of receptors has seven immunoglobulin like domains and a hydrophilic insert sequence in the intracellular tyrosine kinase region. VEGF plays an important role in the proliferation, migration, and vascularization of endothelial cells as the most powerful vascular penetrant and endothelium-specific mitotic source. There is significant positive correlation between the VEGFR expression level and the degree of vascularization and malignancy of tumor tissue. Among them, VEGFR2 is the most important in mediating the biological effect of VEGF, which is closely related to cell chemotaxis and cell division.

Tyrosine kinase inhibitors block the action of tyrosine kinase enzymes. Development of kinase inhibitors for the treatment of cancer has proven successful, with protein kinases now the second most targeted group of drug targets. Over thirty kinase inhibitors have received FDA approval; over 150 are in clinical trials, and many more are in preclinical development. A recent review of kinase targeted cancer therapies (see “Kinase-targeted cancer therapies: progress, challenges and future directions”, Bhullar, et al, Mol Cancer, 2018, 17, 48) provided FDA-approved kinase inhibitors and their drug targets, summarized in Table 1.

TABLE 1

| | Protein | |
|---|---|---|
| Drug target | substrate | Drug |
| VEGFR family | Tyrosine | Axitinib, Lenvatinib, Nintedanib, Regorafenib, Pazopanib, |
| | | Sorafenib, Sunitinib |
| ALK | Tyrosine | Crizotinib, Ceritinib, Alectinib, Brigatinib |
| BCR-Abl | Tyrosine | Bosutinib, Dasatinib, Imatinib, Nilotinib, Ponatinib |
| BTK | Tyrosine | Ibrutinib |
| c-Met | Tyrosine | Crizotinib, Cabozantinib |
| EGFR family | Tyrosine | Gefitinib, Erlotinib, Lapatinib, Vandetanib, Afatinib, Osimertinib |
| JAK family | Tyrosine | Ruxolitinib, Tofacitinib |
| PDGFR α/β | Tyrosine | Axitinib, Gefitinib, Imatinib, Lenvatinib, Nintedanib, Pazopanib, |
| | | Regorafenib, Sorafenib, Sunitinib |
| RET | Tyrosine | Vandetanib |
| Src family | Tyrosine | Bosutinib, Dasatinib, Ponatinib, Vandetanib |
| MEK1/2 | Dual | Trametinib |
| B-Raf | Ser/thre | Vemurafenib, Dabrafenib |
| CDK family | Ser/thre | Palbociclib, Sorafenib, Ribociclib |

However, many factors complicate and impede the clinical efficacy of these drugs. Specific tumor genetics, tumor microenvironment, drug resistance, and pharmacogenomics determine how useful a compound will be in the treatment of a given cancer and these factors are difficult, if not impossible, to predict. For example, some observed safety issues for approved drugs are presented in Table 2.

TABLE 2

| Safety Profile | Bosutinib | Dasatinib | Erlotinib | Gefitinib | Imatinib | Lapatinib |
|---|---|---|---|---|---|---|
| GI | C | C | C | C | C | C |
| Renal | C | R | C | C | R | |
| Musculoskeletal/ | C | R | | | C | C |
| bone | | | | | | |
| Blood/lymph | C | C | R | | C | |
| system | | | | | | |
| Vascular | | C | | C | R | C |
| Skin | C | C | C | C | C | C |
| CMR | | C | C | C | C | C |
| Central nervous | | R | R | | R | R |
| system | | | | | | |
| Nerve | C | C | C | | C | C |
| Eye | | C | C | C | C | |
| Heart | C | C | | | R | R |
| Lung airways | C | C | C | C | C | C |
| Thyroid | | | | | R | |
| Liver/Bile | C | R | R | C | C | C |

| Safety Profile | Nilotinib | Pazopanib | Ponatinib | Sorafenib | Sunitinib |
|---|---|---|---|---|---|
| GI | R | C | C | R | C |
| Renal | R | C | | R | C |
| Musculoskeletal/ | R | C | C | R | C |
| bone | | | | | |
| Blood/lymph | C | C | C | C | C |
| system | | | | | |
| Vascular | R | C | C | C | C |
| Skin | C | C | C | C | C |
| CMR | C | C | | C | C |
| Central nervous | R | | | R | R |
| system | | | | | |
| Nerve | C | C | C | C | C |
| Eye | C | C | C | | C |
| Heart | C | R | C | R | R |
| Lung airways | C | C | C | R | C |
| Thyroid | | C | | | C |
| Liver/Bile | C | C | C | R | R |

C = common or very common;
R = rare

### CMR, Carcinogenic, Mutagenic and Toxic for Reproductive System

Many more tyrosine kinase inhibitors exist, including but not limited, to afatinib, alectinib, apatinib, axitinib, bosutinib, brigatinib, cabozantinib, canertinib, ceritinib, crizotinib, dabrafenib, danusertib, dasatinib, erlotinib, gefitinib, ibrutinib, imatinib, lapatinib, lenvatinib, linifanib, masitinib, neratinib, nilotinib, nintedanib, orantinib, osimertinib, palbociclib, pazopanib, ponatinib, quizartinib, rebastinib, regorafenib, ribociclib, rivoceranib, ruxolitinib, sapitinib, semaxinib, sorafenib, sunitinib, tandutinib, tofacitinib, trametinib, vandetanib, vatalanib, vemurafenib and vismodegib.

VEGF/VEGFR inhibitors include but are not limited to aflibercept, allantoin, lenvatinib, pazopanib, pegaptanib, ramucirumab, ranibizumab, sunitinib, tivozanib, and vandetanib.

### Rivoceranib

Rivoceranib (chemical name N-[4-(1-cyanocyclopentyl)phenyl]-2-{[(pyridin-4-yl)methyl]amino} pyridine-3-carboxamide, also known as YN968D1, developed in China as apatinib and marketed as Aitan®) is an orally administered small molecule tyrosine kinase inhibitor. It selectively inhibits vascular endothelial growth factor receptor (VEGFR)-2 leading to blockage of tumor vascular angiogenesis, diminishes survival of existing blood vessels, and retards growth of tumors. Proliferation of endothelial cells is targeted directly, and inhibition of the release of proangiogenic growth factors by cancer or stromal cells is targeted indirectly.

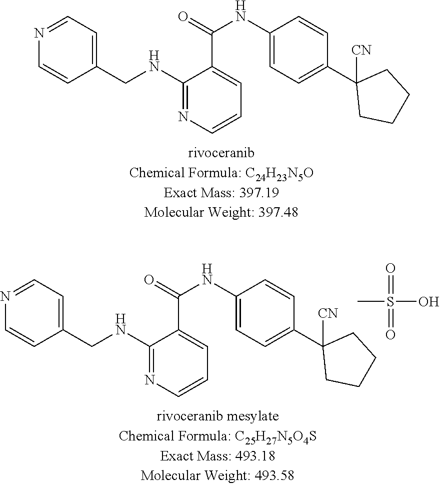

Nonclinical studies completed with rivoceranib demonstrated:
Rivoceranib selectively binds to VEGFR-2 and is efficacious in various tumor bearing animal models.
There are minimal side effects in animals at efficacious doses of rivoceranib.
Rivoceranib is high binding compound with over 97% binding to plasma albumin.
Rivoceranib is generally poorly absorbed.
Steady state levels of Rivoceranib were achieved in approximately 7 days; further accumulation was not evident in studies up to 28-days.

Rivoceranib has been clinically tested in over 1,000 patients and has demonstrated efficacy in numerous cancers including gastric cancer, colorectal cancer (CRC), hepatocellular carcinoma (HCC), non-small-cell lung cancer (NSCLC), esophageal cancer, thyroid cancer, mesothelioma, and neuroendocrine tumors. Several clinical studies of rivoceranib have been completed and are briefly described below.

A Phase 1 study (46 patients) revealed a once daily dose-limiting toxicity of 805 mg rivoceranib (1000 mg rivoceranib mesylate) and a maximum tolerated dose of 685 mg (850 mg mesylate salt). Partial response was noted in 7 patients (19%), stable disease in 24 patients (65%), and a disease control rate of 84% at 8 weeks.

A Phase 1/2a dose escalation and PK study provided a recommended Phase 2a dose of 685 mg (850 mg mesylate salt), where thirty patients then received up to 685 mg rivoceranib (850 mg mesylate salt) in 28-Day cycles (2 cycles). 5 deaths were reported during the study, though a clinical disease control rate was achieved for 93% of patients (n=28 evaluable patients).

A Phase 1 study to evaluate the PK of rivoceranib with and without food for two doses of rivoceranib mesylate (100 mg and 250 mg rivoceranib mesylate, corresponding to 81 mg and 201 mg freebase, respectively) administered in healthy volunteers. Food effects on the bioavailability of the 81 mg rivoceranib dose were minimal, while more pronounced (20-30% increase in bioavailability) for the 201 mg dose.

A Phase 1 study was conducted to evaluate a single dose of rivoceranib mesylate in healthy male patients of Caucasian, Japanese and Chinese origin. The results showed that Cmax and AUC0-∞ in Chinese and Japanese subjects were slightly higher compared to Caucasian subjects, while t_{1/2} values were similar (7.5-8 hours) amongst the three groups.

A Phase 2 study of patients with advanced or metastatic gastric cancer after failure of 2 lines of chemotherapy was completed, where rivoceranib was dosed daily at 685 mg (850 mg rivoceranib mesylate). This study provided placebo-controlled evidence that rivoceranib has significant activity against gastric cancer with a manageable safety profile. In a follow-up to this study, a Phase 3 multi-center, randomized, double-blind, and placebo-controlled study was conducted. In this study, efficacy, median overall survival (OS) and median progression-free survival (PFS) were prolonged in the rivoceranib group compared to placebo. The recommended dose for clinical use was 685 mg rivoceranib (850 mg mesylate salt) once daily. Treatment with rivoceranib was generally well tolerated with most of the adverse reactions manageable by dose interruptions or reductions. Grade 3/4 adverse reactions that occurred in more than 2% of patients were hypertension, HFS, proteinuria, fatigue, anorexia, and elevated aminotransferase.

In December 2014, Rivoceranib received approval in China, for treatment of advanced gastric cancer, and has received orphan medicinal product designation for the treatment of gastric cancer from Europe, the FDA, and the MFDS in South Korea. However, according to a recent review (see L. J. Scott, “Apatinib: A Review in Advanced Gastric Cancer and Other Advanced Cancers,” Drugs, 2018, 78(7), 747-758), “further clinical experience and long-term pharmacovigilance data are required to more definitively establish the efficacy and safety profile of apatinib, including its use in combination with other chemotherapy agents and its role in the management of other types of advanced or metastatic solid tumors”.

In some embodiments, the methods for treating diseases comprise administering a combination of two or more therapies, wherein one of the therapies is a tyrosine kinase inhibitor, or a pharmaceutically acceptable salt thereof.

In some embodiments, the tyrosine kinase inhibitor is administered in the form of a free base. In some embodiments, the tyrosine kinase inhibitor is administered in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts also include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentaflu oropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt.

In some embodiments, the tyrosine kinase inhibitor selective vascular endothelial growth factor receptor-2 (VEGFR2) inhibitor. In some embodiments, the tyrosine kinase inhibitor isafatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of at least 10 mg. In some embodiments, the tyrosine kinase inhibitor is administered in an amount of at least 50 mg. In some embodiments, the tyrosine kinase inhibitor is administered in an amount of at least 100 mg. In some embodiments, the tyrosine kinase inhibitor is administered in an amount of at least 150 mg. In some embodiments, the tyrosine kinase inhibitor is administered in an amount of at least 200 mg. In some embodiments, the tyrosine kinase inhibitor is administered in an amount of at least 225 mg. In some embodiments, the tyrosine kinase inhibitor is administered in an amount of at least 250 mg. In some embodiments, the tyrosine kinase inhibitor is administered in an amount of at least 275 mg. In some embodiments, the tyrosine kinase inhibitor is administered in an amount of at least 300 mg. In some embodiments, the tyrosine kinase inhibitor is administered in an amount of at least 310 mg. In some embodiments, the tyrosine kinase inhibitor is administered in an amount of at least 320 mg. In some embodiments, the tyrosine kinase inhibitor is administered in an amount of at least 325 mg. In some embodiments, the tyrosine kinase inhibitor is administered in an amount of at least 330 mg. In some embodiments, the tyrosine kinase inhibitor is administered in an amount of at least 340 mg. In some embodiments, the tyrosine kinase inhibitor is administered in an amount of at least 350 mg. In some embodiments, the tyrosine kinase inhibitor is administered in an amount of at least 360 mg. In some embodiments, the tyrosine kinase inhibitor is administered in an amount of at least 370 mg. In some embodiments, the tyrosine kinase inhibitor is administered in an amount of at least 375 mg. In some embodiments, the tyrosine kinase inhibitor is administered in an amount of at least 380 mg. In some embodiments, the tyrosine kinase inhibitor is administered in an amount of at least 400 mg. In some embodiments, the tyrosine kinase inhibitor is administered in an amount of at least 410 mg. In some embodiments, the tyrosine kinase inhibitor is administered in an amount of at least 420 mg. In some embodiments, the tyrosine kinase inhibitor is administered in an amount of at least 425 mg. In some embodiments, the tyrosine kinase inhibitor is administered in an amount of at least 430 mg. In some embodiments, the tyrosine kinase inhibitor is administered in an amount of at least 440 mg. In some embodiments, the tyrosine kinase inhibitor is administered in an amount of at least 450 mg. In some embodiments, the tyrosine kinase inhibitor is administered in an amount of at least 460 mg. In some embodiments, the tyrosine kinase inhibitor is administered in an amount of at least 470 mg. In some embodiments, the tyrosine kinase inhibitor is administered in an amount of at least 475 mg. In some embodiments, the tyrosine kinase inhibitor is administered in an amount of at least 480 mg. In some embodiments, the tyrosine kinase inhibitor is administered in an amount of at least 490 mg. In some embodiments, the tyrosine kinase inhibitor is administered in an amount of at least 500 mg. In some embodiments, the tyrosine kinase inhibitor is administered in an amount of at least 525 mg. In some embodiments, the tyrosine kinase inhibitor is administered in an amount of at least 550 mg. In some embodiments, the tyrosine kinase inhibitor is administered in an amount of at least 575 mg. In some embodiments, the tyrosine kinase inhibitor is administered in an amount of at least 600 mg. In some embodiments, the tyrosine kinase inhibitor is administered in an amount of at least 625 mg. In some embodiments, the tyrosine kinase inhibitor is administered in an amount of at least 650 mg. In some embodiments, the tyrosine kinase inhibitor is administered in an amount of at least 675 mg. In some embodiments, the tyrosine kinase inhibitor is administered in an amount of at least 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of about 10 mg. In some embodiments, the tyrosine kinase inhibitor is administered in an amount of about 50 mg. In some embodiments, the tyrosine kinase inhibitor is administered in an amount of about 100 mg. In some embodiments, the tyrosine kinase inhibitor is administered in an amount of about 150 mg. In some embodiments, the tyrosine kinase inhibitor is administered in an amount of about 200 mg. In some embodiments, the tyrosine kinase inhibitor is administered in an amount of about 225 mg. In some embodiments, the tyrosine kinase inhibitor is administered in an amount of about 250 mg. In some embodiments, the tyrosine kinase inhibitor is administered in an amount of about 275 mg. In some embodiments, the tyrosine kinase inhibitor is administered in an amount of about 300 mg. In some embodiments, the tyrosine kinase inhibitor is administered in an amount of about 310 mg. In some embodiments, the tyrosine kinase inhibitor is administered in an amount of about 320 mg. In some embodiments, the tyrosine kinase inhibitor is administered in an amount of about 325 mg. In some embodiments, the tyrosine kinase inhibitor is administered in an amount of about 330 mg. In some embodiments, the tyrosine kinase inhibitor is administered in an amount of about 340 mg. In some embodiments, the tyrosine kinase inhibitor is administered in an amount of about 350 mg. In some embodiments, the tyrosine kinase inhibitor is administered in an amount of about 360 mg. In some embodiments, the tyrosine kinase inhibitor is administered in an amount of about 370 mg. In some embodiments, the tyrosine kinase inhibitor is administered in an amount of about 375 mg. In some embodiments, the tyrosine kinase inhibitor is administered in an amount of about 380 mg. In some embodiments, the tyrosine kinase inhibitor is administered in an amount of about 400 mg. In some embodiments, the tyrosine kinase inhibitor is administered in an amount of about 410 mg. In some embodiments, the tyrosine kinase inhibitor is administered in an amount of about 420 mg. In some embodiments, the tyrosine kinase inhibitor is administered in an amount of about 425 mg. In some embodiments, the tyrosine kinase inhibitor is administered in an amount of about 430 mg. In some embodiments, the tyrosine kinase inhibitor is administered in an amount of about 440 mg. In some embodiments, the tyrosine kinase inhibitor is administered in an amount of about 450 mg. In some embodiments, the tyrosine kinase inhibitor is administered in an amount of about 460 mg. In some embodiments, the tyrosine kinase inhibitor is administered in an amount of about 470 mg. In some embodiments, the tyrosine kinase inhibitor is administered in an amount of about 475 mg. In some embodiments, the tyrosine kinase inhibitor is administered in an amount of about 480 mg. In some embodiments, the tyrosine kinase inhibitor is administered in an amount of about 490 mg. In some embodiments, the tyrosine kinase inhibitor is administered in an amount of about 500 mg. In some embodiments, the tyrosine kinase inhibitor is administered in an amount of about 525 mg. In some embodiments, the tyrosine kinase inhibitor is administered in an amount of about 550 mg. In some embodiments, the tyrosine kinase inhibitor is administered in an amount of about 575 mg. In some embodiments, the tyrosine kinase inhibitor is administered in an amount of about 600 mg. In some embodiments, the tyrosine kinase inhibitor is administered in an amount of about 625 mg. In some embodiments, the tyrosine kinase inhibitor is administered in an amount of about 650 mg. In some embodiments, the tyrosine kinase inhibitor is administered in an amount of about 675 mg. In some embodiments, the tyrosine kinase inhibitor is administered in an amount of about 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, rivoceranib is administered in an amount of about 10 mg. In some embodiments, rivoceranib is administered in an amount of about 50 mg. In some embodiments, rivoceranib is administered in an amount of about 100 mg. In some embodiments, rivoceranib is administered in an amount of about 150 mg. In some embodiments, rivoceranib is administered in an amount of about 200 mg. In some embodiments, rivoceranib is administered in an amount of about 225 mg. In some embodiments, rivoceranib is administered in an amount of about 250 mg. In some embodiments, rivoceranib is administered in an amount of about 275 mg. In some embodiments, rivoceranib is administered in an amount of about 300 mg. In some embodiments, rivoceranib is administered in an amount of about 310 mg. In some embodiments, rivoceranib is administered in an amount of about 320 mg. In some embodiments, rivoceranib is administered in an amount of about 325 mg. In some embodiments, rivoceranib is administered in an amount of about 330 mg. In some embodiments, rivoceranib is administered in an amount of about 340 mg. In some embodiments, rivoceranib is administered in an amount of about 350 mg. In some embodiments, rivoceranib is administered in an amount of about 360 mg. In some embodiments, rivoceranib is administered in an amount of about 370 mg. In some embodiments, rivoceranib is administered in an amount of about 375 mg. In some embodiments, rivoceranib is administered in an amount of about 380 mg. In some embodiments, rivoceranib is administered in an amount of about 400 mg. In some embodiments, rivoceranib is administered in an amount of about 410 mg. In some embodiments, rivoceranib is administered in an amount of about 420 mg. In some embodiments, rivoceranib is administered in an amount of about 425 mg. In some embodiments, rivoceranib is administered in an amount of about 430 mg. In some embodiments, rivoceranib is administered in an amount of about 440 mg. In some embodiments, rivoceranib is administered in an amount of about 450 mg. In some embodiments, rivoceranib is administered in an amount of about 460 mg. In some embodiments, rivoceranib is administered in an amount of about 470 mg. In some embodiments, rivoceranib is administered in an amount of about 475 mg. In some embodiments, rivoceranib is administered in an amount of about 480 mg. In some embodiments, rivoceranib is administered in an amount of about 490 mg. In some embodiments, rivoceranib is administered in an amount of about 500 mg. In some embodiments, rivoceranib is administered in an amount of about 525 mg. In some embodiments, rivoceranib is administered in an amount of about 550 mg. In some embodiments, rivoceranib is administered in an amount of about 575 mg. In some embodiments, rivoceranib is administered in an amount of about 600 mg. In some embodiments, rivoceranib is administered in an amount of about 625 mg. In some embodiments, rivoceranib is administered in an amount of about 650 mg. In some embodiments, rivoceranib is administered in an amount of about 675 mg. In some embodiments, rivoceranib is administered in an amount of about 700 mg.

In some embodiments, rivoceranib mesylate is administered in an amount of about 10 mg. In some embodiments, rivoceranib mesylate is administered in an amount of about 50 mg. In some embodiments, rivoceranib mesylate is administered in an amount of about 100 mg. In some embodiments, rivoceranib mesylate is administered in an amount of about 150 mg. In some embodiments, rivoceranib mesylate is administered in an amount of about 200 mg. In some embodiments, rivoceranib mesylate is administered in an amount of about 225 mg. In some embodiments, rivoceranib mesylate is administered in an amount of about 250 mg. In some embodiments, rivoceranib mesylate is administered in an amount of about 275 mg. In some embodiments, rivoceranib mesylate is administered in an amount of about 300 mg. In some embodiments, rivoceranib mesylate is administered in an amount of about 310 mg. In some embodiments, rivoceranib mesylate is administered in an amount of about 320 mg. In some embodiments, rivoceranib mesylate is administered in an amount of about 325 mg. In some embodiments, rivoceranib mesylate is administered in an amount of about 330 mg. In some embodiments, rivoceranib mesylate is administered in an amount of about 340 mg. In some embodiments, rivoceranib mesylate is administered in an amount of about 350 mg. In some embodiments, rivoceranib mesylate is administered in an amount of about 360 mg. In some embodiments, rivoceranib mesylate is administered in an amount of about 370 mg. In some embodiments, rivoceranib mesylate is administered in an amount of about 375 mg. In some embodiments, rivoceranib mesylate is administered in an amount of about 380 mg. In some embodiments, rivoceranib mesylate is administered in an amount of about 400 mg. In some embodiments, rivoceranib mesylate is administered in an amount of about 410 mg. In some embodiments, rivoceranib mesylate is administered in an amount of about 420 mg. In some embodiments, rivoceranib mesylate is administered in an amount of about 425 mg. In some embodiments, rivoceranib mesylate is administered in an amount of about 430 mg. In some embodiments, rivoceranib mesylate is administered in an amount of about 440 mg. In some embodiments, rivoceranib mesylate is administered in an amount of about 450 mg. In some embodiments, rivoceranib mesylate is administered in an amount of about 460 mg. In some embodiments, rivoceranib mesylate is administered in an amount of about 470 mg. In some embodiments, rivoceranib mesylate is administered in an amount of about 475 mg. In some embodiments, rivoceranib mesylate is administered in an amount of about 480 mg. In some embodiments, rivoceranib mesylate is administered in an amount of about 490 mg. In some embodiments, rivoceranib mesylate is administered in an amount of about 500 mg. In some embodiments, rivoceranib mesylate is administered in an amount of about 525 mg. In some embodiments, rivoceranib mesylate is administered in an amount of about 550 mg. In some embodiments, rivoceranib mesylate is administered in an amount of about 575 mg. In some embodiments, rivoceranib mesylate is administered in an amount of about 600 mg. In some embodiments, rivoceranib mesylate is administered in an amount of about 625 mg. In some embodiments, rivoceranib mesylate is administered in an amount of about 650 mg. In some embodiments, rivoceranib mesylate is administered in an amount of about 675 mg. In some embodiments, rivoceranib mesylate is administered in an amount of about 700 mg.

In some embodiments, the tyrosine kinase inhibitor is administered in an amount of no more than 10 mg. In some embodiments, the tyrosine kinase inhibitor is administered in an amount of no more than 50 mg. In some embodiments, the tyrosine kinase inhibitor is administered in an amount of no more than 100 mg. In some embodiments, the tyrosine kinase inhibitor is administered in an amount of no more than 150 mg. In some embodiments, the tyrosine kinase inhibitor is administered in an amount of no more than 200 mg. In some embodiments, the tyrosine kinase inhibitor is administered in an amount of no more than 225 mg. In some embodiments, the tyrosine kinase inhibitor is administered in an amount of no more than 250 mg. In some embodiments, the tyrosine kinase inhibitor is administered in an amount of no more than 275 mg. In some embodiments, the tyrosine kinase inhibitor is administered in an amount of no more than 300 mg. In some embodiments, the tyrosine kinase inhibitor is administered in an amount of no more than 310 mg. In some embodiments, the tyrosine kinase inhibitor is administered in an amount of no more than 320 mg. In some embodiments, the tyrosine kinase inhibitor is administered in an amount of no more than 325 mg. In some embodiments, the tyrosine kinase inhibitor is administered in an amount of no more than 330 mg. In some embodiments, the tyrosine kinase inhibitor is administered in an amount of no more than 340 mg. In some embodiments, the tyrosine kinase inhibitor is administered in an amount of no more than 350 mg. In some embodiments, the tyrosine kinase inhibitor is administered in an amount of no more than 360 mg. In some embodiments, the tyrosine kinase inhibitor is administered in an amount of no more than 370 mg. In some embodiments, the tyrosine kinase inhibitor is administered in an amount of no more than 375 mg. In some embodiments, the tyrosine kinase inhibitor is administered in an amount of no more than 380 mg. In some embodiments, the tyrosine kinase inhibitor is administered in an amount of no more than 400 mg. In some embodiments, the tyrosine kinase inhibitor is administered in an amount of no more than 410 mg. In some embodiments, the tyrosine kinase inhibitor is administered in an amount of no more than 420 mg. In some embodiments, the tyrosine kinase inhibitor is administered in an amount of no more than 425 mg. In some embodiments, the tyrosine kinase inhibitor is administered in an amount of no more than 430 mg. In some embodiments, the tyrosine kinase inhibitor is administered in an amount of no more than 440 mg. In some embodiments, the tyrosine kinase inhibitor is administered in an amount of no more than 450 mg. In some embodiments, the tyrosine kinase inhibitor is administered in an amount of no more than 460 mg. In some embodiments, the tyrosine kinase inhibitor is administered in an amount of no more than 470 mg. In some embodiments, the tyrosine kinase inhibitor is administered in an amount of no more than 475 mg. In some embodiments, the tyrosine kinase inhibitor is administered in an amount of no more than 480 mg. In some embodiments, the tyrosine kinase inhibitor is administered in an amount of no more than 490 mg. In some embodiments, the tyrosine kinase inhibitor is administered in an amount of no more than 500 mg. In some embodiments, the tyrosine kinase inhibitor is administered in an amount of no more than 525 mg. In some embodiments, the tyrosine kinase inhibitor is administered in an amount of no more than 550 mg. In some embodiments, the tyrosine kinase inhibitor is administered in an amount of no more than 575 mg. In some embodiments, the tyrosine kinase inhibitor is administered in an amount of no more than 600 mg. In some embodiments, the tyrosine kinase inhibitor is administered in an amount of no more than 625 mg. In some embodiments, the tyrosine kinase inhibitor is administered in an amount of no more than 650 mg. In some embodiments, the tyrosine kinase inhibitor is administered in an amount of no more than 675 mg. In some embodiments, the tyrosine kinase inhibitor is administered in an amount of no more than 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the tyrosine kinase inhibitor is administered in an amount of from 100 mg to 900 mg. In some embodiments, the tyrosine kinase inhibitor is administered in an amount of from 150 mg to 850 mg. In some embodiments, the tyrosine kinase inhibitor is administered in an amount of from 175 mg to 825 mg. In some embodiments, the tyrosine kinase inhibitor is administered in an amount of from 200 mg to 800 mg. In some embodiments, the tyrosine kinase inhibitor is administered in an amount of from 225 mg to 775 mg. In some embodiments, the tyrosine kinase inhibitor is administered in an amount of from 250 mg to 750 mg. In some embodiments, the tyrosine kinase inhibitor is administered in an amount of from 250 mg to 725 mg. In some embodiments, the tyrosine kinase inhibitor is administered in an amount of from 250 mg to 700 mg. In some embodiments, the tyrosine kinase inhibitor is administered in an amount of from 250 mg to 675 mg. In some embodiments, the tyrosine kinase inhibitor is administered in an amount of from 250 mg to 650 mg. In some embodiments, the tyrosine kinase inhibitor is administered in an amount of from 250 mg to 600 mg. In some embodiments, the tyrosine kinase inhibitor is administered in an amount of from 250 mg to 550 mg. In some embodiments, the tyrosine kinase inhibitor is administered in an amount of from 250 mg to 500 mg. In some embodiments, the tyrosine kinase inhibitor is administered in an amount of from 250 mg to 450 mg. In some embodiments, the tyrosine kinase inhibitor is administered in an amount of from 275 mg to 425 mg. In some embodiments, the tyrosine kinase inhibitor is administered in an amount of from 300 mg to 600 mg. In some embodiments, the tyrosine kinase inhibitor is administered in an amount of from 300 mg to 550 mg. In some embodiments, the tyrosine kinase inhibitor is administered in an amount of from 300 mg to 500 mg. In some embodiments, the tyrosine kinase inhibitor is administered in an amount of from 300 mg to 450 mg. In some embodiments, the tyrosine kinase inhibitor is administered in an amount of from 300 mg to 400 mg. In some embodiments, the tyrosine kinase inhibitor is administered in an amount of from 350 mg to 450 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate

In some embodiments, rivoceranib is administered in an amount of less than 685 mg.

In some embodiments, rivoceranib mesylate is administered in an amount of less than 685 mg.

In some embodiments, the total daily dose of the tyrosine kinase inhibitor is about 10 mg. In some embodiments, the total daily dose of the tyrosine kinase inhibitor is about 50 mg. In some embodiments, the total daily dose of the tyrosine kinase inhibitor is about 100 mg. In some embodiments, the total daily dose of the tyrosine kinase inhibitor is about 150 mg. In some embodiments, the total daily dose of the tyrosine kinase inhibitor is about 200 mg. In some embodiments, the total daily dose of the tyrosine kinase inhibitor is about 225 mg. In some embodiments, the total daily dose of the tyrosine kinase inhibitor is about 250 mg. In some embodiments, the total daily dose of the tyrosine kinase inhibitor is about 275 mg. In some embodiments, the total daily dose of the tyrosine kinase inhibitor is about 300 mg. In some embodiments, the total daily dose of the tyrosine kinase inhibitor is about 310 mg. In some embodiments, the total daily dose of the tyrosine kinase inhibitor is about 320 mg. In some embodiments, the total daily dose of the tyrosine kinase inhibitor is about 325 mg. In some embodiments, the total daily dose of the tyrosine kinase inhibitor is about 330 mg. In some embodiments, the total daily dose of the tyrosine kinase inhibitor is about 340 mg. In some embodiments, the total daily dose of the tyrosine kinase inhibitor is about 350 mg. In some embodiments, the total daily dose of the tyrosine kinase inhibitor is about 360 mg. In some embodiments, the total daily dose of the tyrosine kinase inhibitor is about 370 mg. In some embodiments, the total daily dose of the tyrosine kinase inhibitor is about 375 mg. In some embodiments, the total daily dose of the tyrosine kinase inhibitor is about 380 mg. In some embodiments, the total daily dose of the tyrosine kinase inhibitor is about 400 mg. In some embodiments, the total daily dose of the tyrosine kinase inhibitor is about 410 mg. In some embodiments, the total daily dose of the tyrosine kinase inhibitor is about 420 mg. In some embodiments, the total daily dose of the tyrosine kinase inhibitor is about 425 mg. In some embodiments, the total daily dose of the tyrosine kinase inhibitor is about 430 mg. In some embodiments, the total daily dose of the tyrosine kinase inhibitor is about 440 mg. In some embodiments, the total daily dose of the tyrosine kinase inhibitor is about 450 mg. In some embodiments, the total daily dose of the tyrosine kinase inhibitor is about 460 mg. In some embodiments, the total daily dose of the tyrosine kinase inhibitor is about 470 mg. In some embodiments, the total daily dose of the tyrosine kinase inhibitor is about 475 mg. In some embodiments, the total daily dose of the tyrosine kinase inhibitor is about 480 mg. In some embodiments, the total daily dose of the tyrosine kinase inhibitor is about 490 mg. In some embodiments, the total daily dose of the tyrosine kinase inhibitor is about 500 mg. In some embodiments, the total daily dose of the tyrosine kinase inhibitor is about 525 mg. In some embodiments, the total daily dose of the tyrosine kinase inhibitor is about 550 mg. In some embodiments, the total daily dose of the tyrosine kinase inhibitor is about 575 mg. In some embodiments, the total daily dose of the tyrosine kinase inhibitor is about 600 mg. In some embodiments, the total daily dose of the tyrosine kinase inhibitor is about 625 mg. In some embodiments, the total daily dose of the tyrosine kinase inhibitor is about 650 mg. In some embodiments, the total daily dose of the tyrosine kinase inhibitor is about 675 mg. In some embodiments, the total daily dose of the tyrosine kinase inhibitor is about 700 mg. In some embodiments, the tyrosine kinase inhibitor is rivoceranib. In some embodiments, the tyrosine kinase inhibitor is a pharmaceutically acceptable salt of rivoceranib. In some embodiments, the tyrosine kinase inhibitor is rivoceranib mesylate.

In some embodiments, the total daily dose of rivoceranib is about 10 mg. In some embodiments, the total daily dose of rivoceranib is about 50 mg. In some embodiments, the total daily dose of rivoceranib is about 100 mg. In some embodiments, the total daily dose of rivoceranib is about 150 mg. In some embodiments, the total daily dose of rivoceranib is about 200 mg. In some embodiments, the total daily dose of rivoceranib is about 225 mg. In some embodiments, the total daily dose of rivoceranib is about 250 mg. In some embodiments, the total daily dose of rivoceranib is about 275 mg. In some embodiments, the total daily dose of rivoceranib is about 300 mg. In some embodiments, the total daily dose of rivoceranib is about 310 mg. In some embodiments, the total daily dose of rivoceranib is about 320 mg. In some embodiments, the total daily dose of rivoceranib is about 325 mg. In some embodiments, the total daily dose of rivoceranib is about 330 mg. In some embodiments, the total daily dose of rivoceranib is about 340 mg. In some embodiments, the total daily dose of rivoceranib is about 350 mg. In some embodiments, the total daily dose of rivoceranib is about 360 mg. In some embodiments, the total daily dose of rivoceranib is about 370 mg. In some embodiments, the total daily dose of rivoceranib is about 375 mg. In some embodiments, the total daily dose of rivoceranib is about 380 mg. In some embodiments, the total daily dose of rivoceranib is about 400 mg. In some embodiments, the total daily dose of rivoceranib is about 410 mg. In some embodiments, the total daily dose of rivoceranib is about 420 mg. In some embodiments, the total daily dose of rivoceranib is about 425 mg. In some embodiments, the total daily dose of rivoceranib is about 430 mg. In some embodiments, the total daily dose of rivoceranib is about 440 mg. In some embodiments, the total daily dose of rivoceranib is about 450 mg. In some embodiments, the total daily dose of rivoceranib is about 460 mg. In some embodiments, the total daily dose of rivoceranib is about 470 mg. In some embodiments, the total daily dose of rivoceranib is about 475 mg. In some embodiments, the total daily dose of rivoceranib is about 480 mg. In some embodiments, the total daily dose of rivoceranib is about 490 mg. In some embodiments, the total daily dose of rivoceranib is about 500 mg. In some embodiments, the total daily dose of rivoceranib is about 525 mg. In some embodiments, the total daily dose of rivoceranib is about 550 mg. In some embodiments, the total daily dose of rivoceranib is about 575 mg. In some embodiments, the total daily dose of rivoceranib is about 600 mg. In some embodiments, the total daily dose of rivoceranib is about 625 mg. In some embodiments, the total daily dose of rivoceranib is about 650 mg. In some embodiments, the total daily dose of rivoceranib is about 675 mg. In some embodiments, the total daily dose of rivoceranib is about 700 mg.

In some embodiments, the total daily dose of rivoceranib mesylate is about 10 mg. In some embodiments, the total daily dose of rivoceranib mesylate is about 50 mg. In some embodiments, the total daily dose of rivoceranib mesylate is about 100 mg. In some embodiments, the total daily dose of rivoceranib mesylate is about 150 mg. In some embodiments, the total daily dose of rivoceranib mesylate is about 200 mg. In some embodiments, the total daily dose of rivoceranib mesylate is about 225 mg. In some embodiments, the total daily dose of rivoceranib mesylate is about 250 mg. In some embodiments, the total daily dose of rivoceranib mesylate is about 275 mg. In some embodiments, the total daily dose of rivoceranib mesylate is about 300 mg. In some embodiments, the total daily dose of rivoceranib mesylate is about 310 mg. In some embodiments, the total daily dose of rivoceranib mesylate is about 320 mg. In some embodiments, the total daily dose of rivoceranib mesylate is about 325 mg. In some embodiments, the total daily dose of rivoceranib mesylate is about 330 mg. In some embodiments, the total daily dose of rivoceranib mesylate is about 340 mg. In some embodiments, the total daily dose of rivoceranib mesylate is about 350 mg. In some embodiments, the total daily dose of rivoceranib mesylate is about 360 mg. In some embodiments, the total daily dose of rivoceranib mesylate is about 370 mg. In some embodiments, the total daily dose of rivoceranib mesylate is about 375 mg. In some embodiments, the total daily dose of rivoceranib mesylate is about 380 mg. In some embodiments, the total daily dose of rivoceranib mesylate is about 400 mg. In some embodiments, the total daily dose of rivoceranib mesylate is about 410 mg. In some embodiments, the total daily dose of rivoceranib mesylate is about 420 mg. In some embodiments, the total daily dose of rivoceranib mesylate is about 425 mg. In some embodiments, the total daily dose of rivoceranib mesylate is about 430 mg. In some embodiments, the total daily dose of rivoceranib mesylate is about 440 mg. In some embodiments, the total daily dose of rivoceranib mesylate is about 450 mg. In some embodiments, the total daily dose of rivoceranib mesylate is about 460 mg. In some embodiments, the total daily dose of rivoceranib mesylate is about 470 mg. In some embodiments, the total daily dose of rivoceranib mesylate is about 475 mg. In some embodiments, the total daily dose of rivoceranib mesylate is about 480 mg. In some embodiments, the total daily dose of rivoceranib mesylate is about 490 mg. In some embodiments, the total daily dose of rivoceranib mesylate is about 500 mg. In some embodiments, the total daily dose of rivoceranib mesylate is about 525 mg. In some embodiments, the total daily dose of rivoceranib mesylate is about 550 mg. In some embodiments, the total daily dose of rivoceranib mesylate is about 575 mg. In some embodiments, the total daily dose of rivoceranib mesylate is about 600 mg. In some embodiments, the total daily dose of rivoceranib mesylate is about 625 mg. In some embodiments, the total daily dose of rivoceranib mesylate is about 650 mg. In some embodiments, the total daily dose of rivoceranib mesylate is about 675 mg. In some embodiments, the total daily dose of rivoceranib mesylate is about 700 mg.

In some embodiments, the tyrosine kinase inhibitor is administered orally. In some embodiments, the tyrosine kinase inhibitor is administered in an oral liquid, solid or semisolid dosage form. In some embodiments, the tyrosine kinase inhibitor is administered as a solid oral dosage form. In some embodiments, the tyrosine kinase inhibitor is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, the tyrosine kinase inhibitor is administered as a dried powder, a liquid, a capsule, a pellet or a tablet. In some embodiments, the tyrosine kinase inhibitor is administered as a tablet. In some embodiments, the tyrosine kinase inhibitor is administered as a film coated tablet.

In such embodiments, wherein the tyrosine kinase inhibitor is administered as a solid oral dosage form, the tyrosine kinase inhibitor may be admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

In some embodiments, solid dosage forms may be prepared with coatings and shells, such as enteric coatings and others known in the art. They may contain pacifying agents, and can also be of such composition that they release the tyrosine kinase inhibitor in a certain part of the intestinal tract in a delayed manner. Examples of embedded compositions that can be used are polymeric substances and waxes. The tyrosine kinase inhibitor may also be in microencapsulated form, if appropriate, with one or more of the above-mentioned excipients

In some embodiments, the tablet comprises the tyrosine kinase inhibitor in an amount of about 100 mg. In some embodiments, the tablet comprises the tyrosine kinase inhibitor in an amount of about 150 mg. In some embodiments, the tablet comprises the tyrosine kinase inhibitor in an amount of about 200 mg. In some embodiments, the tablet comprises the tyrosine kinase inhibitor in an amount of about 250 mg. In some embodiments, the tablet comprises the tyrosine kinase inhibitor in an amount of about 300 mg. In some embodiments, the tablet comprises the tyrosine kinase inhibitor in an amount of about 350 mg. In some embodiments, the tablet comprises the tyrosine kinase inhibitor in an amount of about 400 mg. In some embodiments, the tablet comprises the tyrosine kinase inhibitor in an amount of about 450 mg. In some embodiments, the tablet comprises the tyrosine kinase inhibitor in an amount of about 500 mg. In some embodiments, the tablet comprises the tyrosine kinase inhibitor in an amount of about 550 mg. In some embodiments, the tablet comprises the tyrosine kinase inhibitor in an amount of about 600 mg. In some embodiments, the tablet comprises the tyrosine kinase inhibitor in an amount of about 650 mg. In some embodiments, the tablet further comprises one or more of pregelatinized starch, microcrystalline cellulose, sodium starch glycolate, povidone (K-29/32), colloidal silicon dioxide, magnesium stearate and Opadry white.

In some embodiments, the tyrosine kinase inhibitor is administered as a liquid oral dosage form. In some embodiments, the tyrosine kinase inhibitor is administered as a solution, suspension, drink, syrup, elixir, ampoule, dispersion, semi-solid or soft gel.

In some embodiments, the tyrosine kinase inhibitor is administered parenterally. In some embodiments, the tyrosine kinase inhibitor is administered intradermaly, subcutaneously, intramuscularly, intraosseously, intraperitoneally or intravenously. In some embodiments, the tyrosine kinase inhibitor is administered intraperitoneally. In some embodiments, the tyrosine kinase inhibitor is administered intravenously.

In some embodiments, rivoceranib is administered orally. In some embodiments, rivoceranib is administered in an oral liquid, solid or semisolid dosage form. In some embodiments, rivoceranib is administered as a solid oral dosage form. In some embodiments, rivoceranib is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, rivoceranib is administered as a dried powder, a liquid, a capsule, a pellet or a tablet. In some embodiments, rivoceranib is administered as a tablet. In some embodiments, rivoceranib is administered as a film coated tablet.

In some embodiments, the tablet comprises rivoceranib in an amount of about 100 mg. In some embodiments, the tablet comprises rivoceranib in an amount of about 150 mg. In some embodiments, the tablet comprises rivoceranib in an amount of about 200 mg. In some embodiments, the tablet comprises rivoceranib in an amount of about 250 mg. In some embodiments, the tablet comprises rivoceranib in an amount of about 300 mg. In some embodiments, the tablet comprises rivoceranib in an amount of about 350 mg. In some embodiments, the tablet comprises rivoceranib in an amount of about 400 mg. In some embodiments, the tablet comprises rivoceranib in an amount of about 450 mg. In some embodiments, the tablet comprises rivoceranib in an amount of about 500 mg. In some embodiments, the tablet comprises rivoceranib in an amount of about 550 mg. In some embodiments, the tablet comprises rivoceranib in an amount of about 600 mg. In some embodiments, the tablet comprises rivoceranib in an amount of about 650 mg. In some embodiments, the tablet further comprises one or more of pregelatinized starch, microcrystalline cellulose, sodium starch glycolate, povidone (K-29/32), colloidal silicon dioxide, magnesium stearate and Opadry white.

In some embodiments, rivoceranib is administered as a liquid oral dosage form. In some embodiments, rivoceranib is administered as a solution, suspension, drink, syrup, elixir, ampoule, dispersion, semi-solid or soft gel.

In some embodiments, rivoceranib is administered parenterally. In some embodiments, rivoceranib is administered intradermaly, subcutaneously, intramuscularly, intraosseously, intraperitoneally or intravenously. In some embodiments, rivoceranib is administered intraperitoneally. In some embodiments, rivoceranib is administered intravenously.

In some embodiments, rivoceranib mesylate is administered orally. In some embodiments, rivoceranib mesylate is administered in an oral liquid, solid or semisolid dosage form. In some embodiments, rivoceranib mesylate is administered as a solid oral dosage form. In some embodiments, rivoceranib mesylate is administered as a pill, tablet, chewable tablet, specialty tablet, buccal tablet, sub-lingual tablet, orally-disintegrating tablet, capsule, gel capsule, soft gel capsule, hard gel capsule, sachet, powder, granule, crystal or orally dispersible film. In some embodiments, rivoceranib mesylate is administered as a dried powder, a liquid, a capsule, a pellet or a tablet. In some embodiments, rivoceranib mesylate is administered as a tablet. In some embodiments, rivoceranib mesylate is administered as a film coated tablet.

In some embodiments, the tablet comprises rivoceranib mesylate in an amount of about 100 mg. In some embodiments, the tablet comprises rivoceranib mesylate in an amount of about 150 mg. In some embodiments, the tablet comprises rivoceranib mesylate in an amount of about 200 mg. In some embodiments, the tablet comprises rivoceranib mesylate in an amount of about 250 mg. In some embodiments, the tablet comprises rivoceranib mesylate in an amount of about 300 mg. In some embodiments, the tablet comprises rivoceranib mesylate in an amount of about 350 mg. In some embodiments, the tablet comprises rivoceranib mesylate in an amount of about 400 mg. In some embodiments, the tablet comprises rivoceranib mesylate in an amount of about 450 mg. In some embodiments, the tablet comprises rivoceranib mesylate in an amount of about 500 mg. In some embodiments, the tablet comprises rivoceranib mesylate in an amount of about 550 mg. In some embodiments, the tablet comprises rivoceranib mesylate in an amount of about 600 mg. In some embodiments, the tablet comprises rivoceranib mesylate in an amount of about 650 mg. In some embodiments, the tablet further comprises one or more of pregelatinized starch, microcrystalline cellulose, sodium starch glycolate, povidone (K-29/32), colloidal silicon dioxide, magnesium stearate and Opadry white.

In some embodiments, rivoceranib mesylate is administered as a liquid oral dosage form. In some embodiments, rivoceranib mesylate is administered as a solution, suspension, drink, syrup, elixir, ampoule, dispersion, semi-solid or soft gel.

In some embodiments, rivoceranib mesylate is administered parenterally. In some embodiments, rivoceranib mesylate is administered intradermaly, subcutaneously, intramuscularly, intraosseously, intraperitoneally or intravenously. In some embodiments, rivoceranib mesylate is administered intraperitoneally. In some embodiments, rivoceranib mesylate is administered intravenously.

In some embodiments, the tyrosine kinase inhibitor is administered once daily. In some embodiments, the tyrosine kinase inhibitor is administered twice daily.

In some embodiments, rivoceranib is administered once daily. In some embodiments, rivoceranib is administered twice daily.

In some embodiments, rivoceranib mesylate is administered once daily. In some embodiments, rivoceranib mesylate is administered twice daily.

### Tubulin Binding Agents

Tubulin binding agents (or tubulin inhibitors) inhibit cell mitosis by binding to tubulin in the mitotic spindle thereby preventing polymerization or depolymerization into the microtubules. Tubulin binding agents may be classified on the basis of their mode of action and binding site:

I. Tubulin polymerization inhibitors, acting at the:
a) Colchicine binding site—examples include colchicine (useful for treating non-neoplastic diseases such as gout flare) combrestatin, 2-methoxyestradiol, and the like.
b) Vinca alkaloid binding site—examples include vinblastine, vincristine, vinorelbine, vinfluine, dolastatins, cryptophysin and the like, useful for treating bladder cancer, breast cancer, Hodgkin's disease, leukemia, lung cancer, non-small-cell lung cancer, lymphomas, testicular cancer, solid tumors, etc.

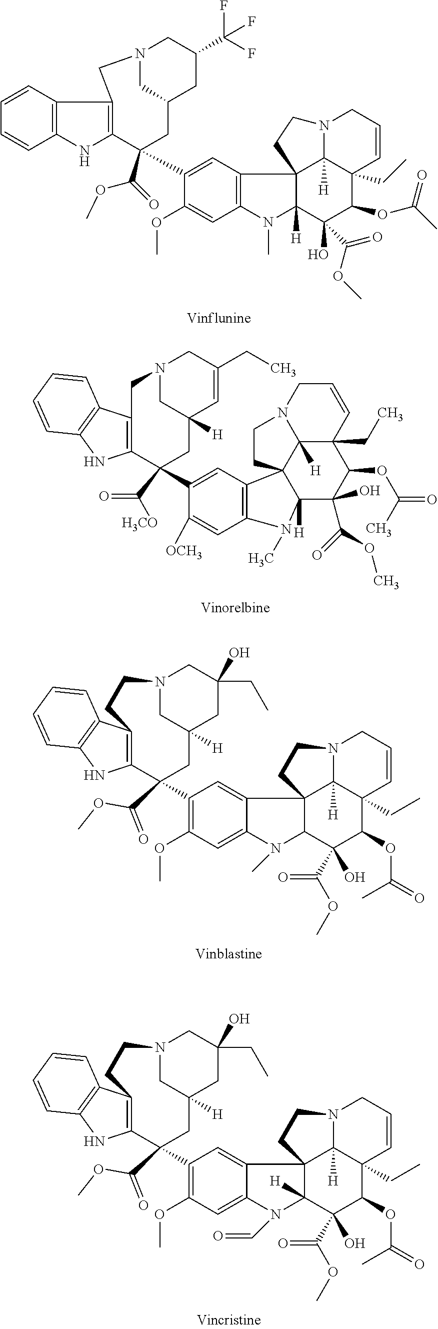

II. Tubulin depolymerization inhibitors, such as the taxanes.

### Taxanes

Taxanes are diterpenoids, useful as chemotherapy agents for their ability to prevent cancer cells from dividing by interfering with the normal function of microtubules during cell division. Taxane-treated cells have defects in mitotic spindle assembly, chromosome segregation and cell division. Taxanes bind to the β subunit of tubulin, creating a hyper-stable microtubule/taxane complex unable to disassemble. This adversely affects cell function as the shortening and lengthening of microtubules (termed dynamic instability) is required for their function as a transportation highway for the cell. Chromosomes rely upon this property of microtubules during mitosis and in the presence of a taxane cannot achieve a metaphase spindle configuration. This blocks the progression of mitosis and prolonged activation of the mitotic checkpoint triggers apoptosis or reversion to the G0-phase of the cell cycle without cell division. Exemplary taxanes that have received regulatory approval for the treatment of diseases include paclitaxel, docetaxel (brand name TAXOTERE®), approved for the treatment of breast cancer, lung cancer, prostate cancer, and head and neck cancer, cabazitaxel (brand name JEVTANA®) is approved to for the treatment of prostate cancer.

Paclitaxel was discovered in 1966 in extracts from Pacific yew and its chemical structure established in 1971. Paclitaxel (brand name Taxol®) received FDA approval by for the treatment of ovarian cancer in 1992, breast cancer in 1994, Kaposi's sarcoma in 1997 and lung cancer in 1998. It is administered by intravenous injection. Exemplary recommended dosing of Paclitaxel (administered as ABRAXANE®) is shown in Table 3.

TABLE 3

| | Dose | Administration |
|---|---|---|
| Cancer type | (mg/m²) | time (mins) |
| Metastatic Breast | 260 | 30 |
| Non-Small Cell Lung | 100 | 30 |
| Pancreatic | 125 | 30-40 |
| Adenocarcinoma | | |

Paclitaxel is available in single-use vials; each containing 100 mg of sterile lyophilized powder containing paclitaxel-albumin bound particles for reconstitution with 20 mL of Sodium Chloride solution (0.9%), to provide 20 mL of reconstituted suspension containing 5 mg/mL paclitaxel.

Dosing volume (mL)=Total dose (mg)/5(mg/mL)

Paclitaxel and docetaxel serve as two of the most important drugs for the treatment of various cancers, however drug resistance imposes limitations to their efficacy. Resistance to taxanes is associated mainly with the increased expression of the multiple multidrug resistance (MIDR) genes that encode various ATP-binding cassette transport proteins such as P-glycoprotein (P-gp) and breast cancer resistance protein (BCRP). Further, common side effects associated with taxane use include hair loss, bone marrow suppression and low white blood cell count, numbness, allergic reactions, muscle pains, vomiting, diarrhea, stomach and intestine problems. Other serious side effects include heart problems, kidney failure, increased risk of infection, and lung inflammation or breathing problems.

In addition to side effects due to paclitaxel, due to poor solubility, the compound, when first approved, was dissolved in Cremophor EL, which is also somewhat toxic. To address this issue, paclitaxel is now available as albumin-bound particles, (marketed as ABRAXANE®). This aids in dissolving paclitaxel (so Cremophor EL is no longer required). There still however remain toxic side effects; in clinical studies, commonly reported ABRAXANE® related side effects included neutropenia (23-82%), hair loss (64-90%), nerve pain (58-68%), fatigue (34-45%), anemia (34%), nausea (20-33%), diarrhea (27%), infections (24%), muscle/joint pain (19-45%), shortness of breath (12%), fluid retention (10%), cough (7%), mouth sores (7%), and bleeding (2%) (occurrence rates in ranges due to differences between clinical studies).

Various taxane analogs include, but are not limited to:

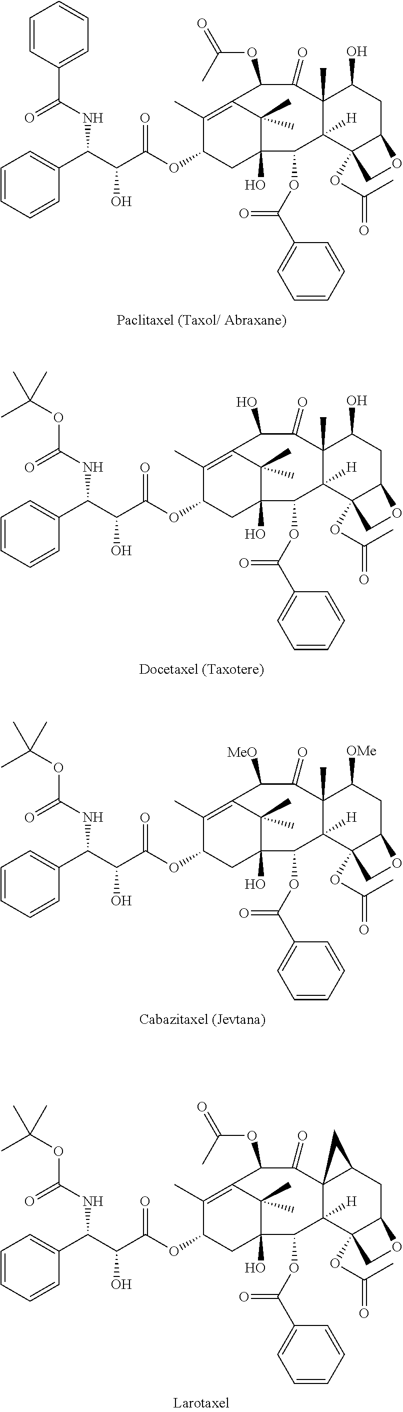

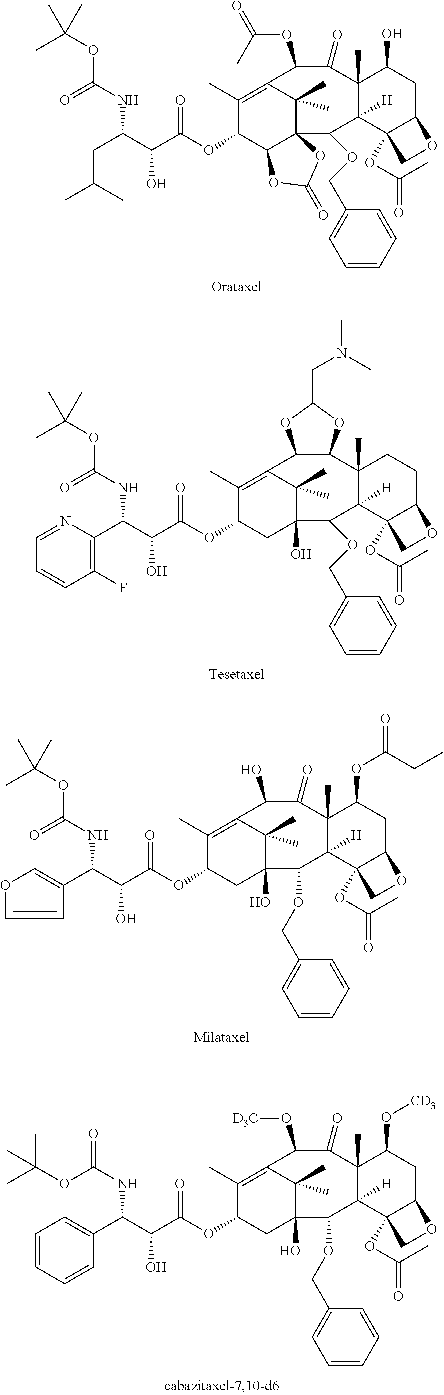

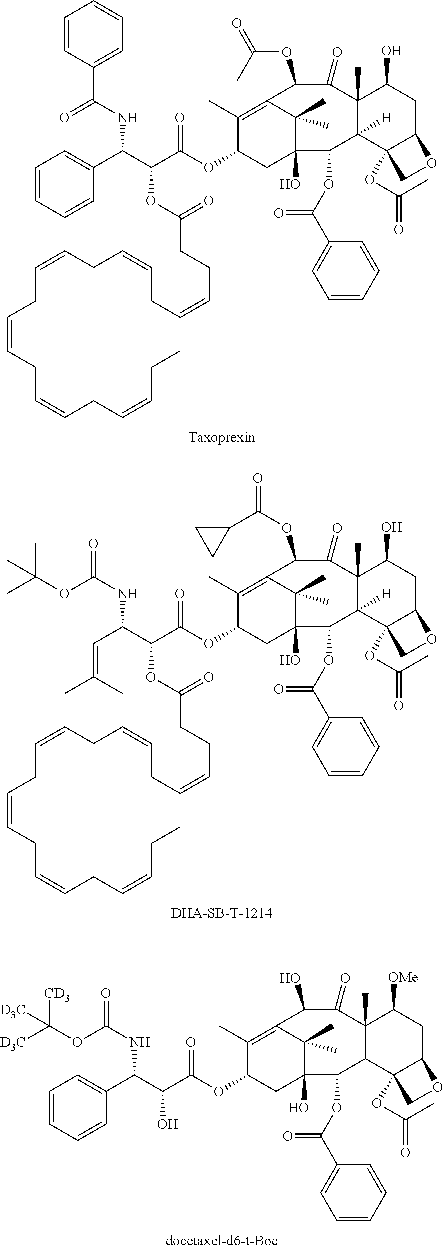

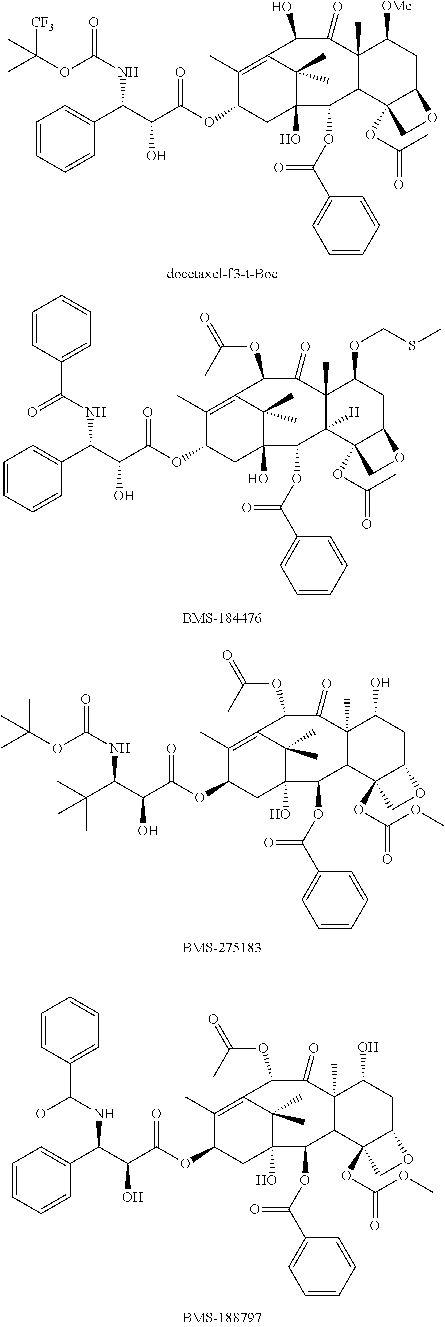

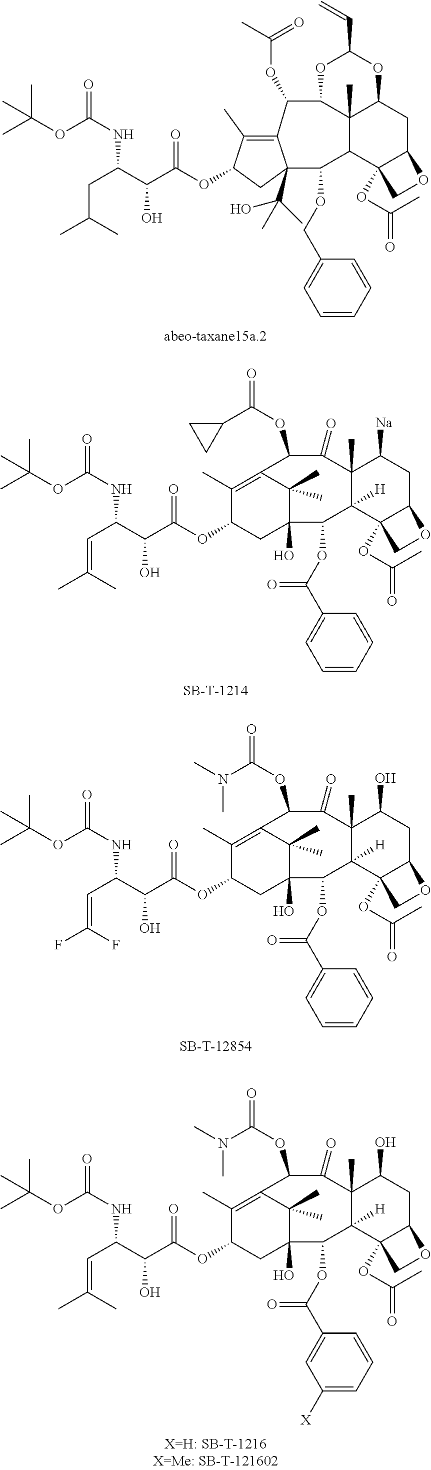

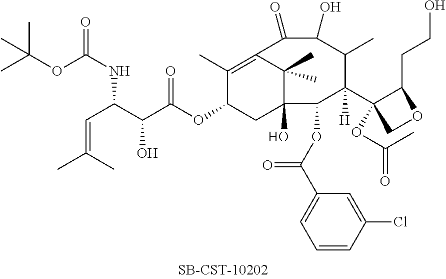

### Body Surface Area and Dosing

In some instances drugs dosages are determined as a factor of patient body surface area (BSA). In some instances BSA is a better indicator of metabolic mass than body weight because it is less affected by abnormal adipose mass, e.g., a patient with a larger BSA would presumably have larger organs for a drug to clear through. Indeed, there can be a 4-10 fold variation in drug clearance between individuals.

Various formulae exist, using height and weight, to calculate BSA without direct measurement. The most widely used is the Du Bois formula, which has been shown to be equally as effective in estimating BSA in obese and non-obese patients.

BSA=0.007184×W^{0.425}×H^{0.725}

where W is mass in kg, and H is height in cm.
The average adult male BSA is 2.060 m². The average adult female BSA is 1.830 m². In some instances, paclitaxel doses are given in units of mg/m².

In some embodiments, the methods for treating diseases comprise administering a combination of two or more therapies, wherein one of the therapies is a tubulin binding agent, or a pharmaceutically acceptable salt thereof. In some embodiments, one of the therapies is a tubulin inhibitor, or a pharmaceutically acceptable salt thereof. In some embodiments, one of the therapies is a tubulin polymerization inhibitor or a pharmaceutically acceptable salt thereof. In some embodiments, one of the therapies is colchicine, combrestatin, 2-methoxyestradiol, vinblastine, vincristine, vinorelbine, vinfluine, dolastatins, cryptophysin or a pharmaceutically acceptable salt thereof. In some embodiments, one of the therapies is a tubulin depolymerization inhibitor or a pharmaceutically acceptable salt thereof. In some embodiments, one of the therapies is a taxane or a pharmaceutically acceptable salt thereof. In some embodiments, one of the therapies is paclitaxel, docetaxel, cabazitaxel, Larotaxel, orataxel, tesetaxel, milataxel, taxoprexin, docetaxel-d₆-t-Boc, docetaxel-f3-t-Boc, cabazitaxel-7,10-d₆, abeo-taxane15a.2, BMS-184476, BMS-188797, BMS-275183, SB-T-1214, SB-T-1216, SB-T-12854, SB-T-121602, SB-CST-10202, DHA-SB-T-1214 or a pharmaceutically acceptable salt thereof. In some embodiments, one of the therapies is paclitaxel, or a pharmaceutically acceptable salt thereof.

In some embodiments, the tubulin binding agent is administered in the form of a free base. In some embodiments, the tubulin binding agent is administered in the form of a pharmaceutically acceptable salt. As used herein, a pharmaceutically acceptable salt includes, but is not limited to, metal salts, such as sodium salts, potassium salts, and lithium salts; alkaline earth metals, such as calcium salts, magnesium salts, and the like; organic amine salts, such as triethylamine salts, pyridine salts, picoline salts, ethanolamine salts, triethanolamine salts, dicyclohexylamine salts, N,N′-dibenzylethylenediamine salts, and the like; inorganic acid salts such as hydrochloride salts, hydrobromide salts, sulfate salts, phosphate salts, and the like; organic acid salts such as formate salts, acetate salts, trifluoroacetate salts, maleate salts, tartrate salts, and the like; sulfonate salts such as methanesulfonate salts, benzenesulfonate salts, p-toluenesulfonate salts, and the like; and amino acid salts, such as arginate salts, asparginate salts, glutamate salts, and the like. Pharmaceutically acceptable salts also include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentaflu oropropionate), bis(pyridine carboxylate), bis(trifluoroacetate), chlorhydrate, and sulfate pentahydrate. Other representative pharmaceutically acceptable salts include, e.g., water-soluble and water-insoluble salts, such as the acetate, amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A hydrate is another example of a pharmaceutically acceptable salt.

In some embodiments, the tubulin binding agent is administered as a composition comprising nanoparticles comprising the tubulin binding agent and albumin. In some embodiments, the tubulin binding agent in the nanoparticles is coated with the albumin. In some embodiments, the nanoparticles have an average diameter of no greater than about 200 nm. In some embodiments, the weight ratio of albumin and tubulin binding agent in the nanoparticle composition is about 9:1. In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel. In some embodiments, the tubulin binding agent is paclitaxel mesylate. In some embodiments, paclitaxel is administered as a composition comprising nanoparticles comprising paclitaxel and albumin. In some embodiments, the paclitaxel in the nanoparticles is coated with the albumin. In some embodiments, the nanoparticles have an average diameter of no greater than about 200 nm. In some embodiments, the weight ratio of albumin and paclitaxel in the nanoparticle composition is about 9:1 In some embodiments, paclitaxel mesylate is administered as a composition comprising nanoparticles comprising paclitaxel and albumin. In some embodiments, the paclitaxel mesylate in the nanoparticles is coated with the albumin. In some embodiments, the nanoparticles have an average diameter of no greater than about 200 nm. In some embodiments, the weight ratio of albumin and paclitaxel mesylate in the nanoparticle composition is about 9:1.

In some embodiments, the tubulin binding agent is administered in an amount of about 25 mg/m². In some embodiments, the tubulin binding agent is administered in an amount of about 30 mg/m². In some embodiments, the tubulin binding agent is administered in an amount of about 35 mg/m². In some embodiments, the tubulin binding agent is administered in an amount of about 40 mg/m². In some embodiments, the tubulin binding agent is administered in an amount of about 45 mg/m². In some embodiments, the tubulin binding agent is administered in an amount of about 50 mg/m². In some embodiments, the tubulin binding agent is administered in an amount of about 55 mg/m². In some embodiments, the tubulin binding agent is administered in an amount of about 60 mg/m². In some embodiments, the tubulin binding agent is administered in an amount of about 65 mg/m². In some embodiments, the tubulin binding agent is administered in an amount of about 70 mg/m². In some embodiments, the tubulin binding agent is administered in an amount of about 75 mg/m². In some embodiments, the tubulin binding agent is administered in an amount of about 80 mg/m². In some embodiments, the tubulin binding agent is administered in an amount of about 85 mg/m². In some embodiments, the tubulin binding agent is administered in an amount of about 90 mg/m². In some embodiments, the tubulin binding agent is administered in an amount of about 95 mg/m². In some embodiments, the tubulin binding agent is administered in an amount of about 100 mg/m². In some embodiments, the tubulin binding agent is administered in an amount of about 105 mg/m². In some embodiments, the tubulin binding agent is administered in an amount of about 110 mg/m². In some embodiments, the tubulin binding agent is administered in an amount of about 115 mg/m². In some embodiments, the tubulin binding agent is administered in an amount of about 120 mg/m². In some embodiments, the tubulin binding agent is administered in an amount of about 125 mg/m². In some embodiments, the tubulin binding agent is administered in an amount of about 130 mg/m². In some embodiments, the tubulin binding agent is administered in an amount of about 135 mg/m². In some embodiments, the tubulin binding agent is administered in an amount of about 140 mg/m². In some embodiments, the tubulin binding agent is administered in an amount of about 145 mg/m². In some embodiments, the tubulin binding agent is administered in an amount of about 150 mg/m². In some embodiments, the tubulin binding agent is administered in an amount of about 160 mg/m². In some embodiments, the tubulin binding agent is administered in an amount of about 170 mg/m². In some embodiments, the tubulin binding agent is administered in an amount of about 175 mg/m². In some embodiments, the tubulin binding agent is administered in an amount of about 180 mg/m². In some embodiments, the tubulin binding agent is administered in an amount of about 190 mg/m². In some embodiments, the tubulin binding agent is administered in an amount of about 200 mg/m². In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel.

In some embodiments, the tubulin binding agent is administered in an amount of no more than 70 mg/m². In some embodiments, the tubulin binding agent is administered in an amount of no more than 80 mg/m². In some embodiments, the tubulin binding agent is administered in an amount of no more than 90 mg/m². In some embodiments, the tubulin binding agent is administered in an amount of no more than 100 mg/m². In some embodiments, the tubulin binding agent is administered in an amount of no more than 125 mg/m². In some embodiments, the tubulin binding agent is administered in an amount of no more than 150 mg/m². In some embodiments, the tubulin binding agent is administered in an amount of no more than 175 mg/m². In some embodiments, the tubulin binding agent is administered in an amount of no more than 200 mg/m². In some embodiments, the tubulin binding agent is administered in an amount of no more than 250 mg/m². In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel.

In some embodiments, paclitaxel is administered in an amount of about 25 mg/m². In some embodiments, paclitaxel is administered in an amount of about 30 mg/m². In some embodiments, paclitaxel is administered in an amount of about 35 mg/m². In some embodiments, paclitaxel is administered in an amount of about 40 mg/m². In some embodiments, paclitaxel is administered in an amount of about 45 mg/m². In some embodiments, paclitaxel is administered in an amount of about 50 mg/m². In some embodiments, paclitaxel is administered in an amount of about 55 mg/m². In some embodiments, paclitaxel is administered in an amount of about 60 mg/m². In some embodiments, paclitaxel is administered in an amount of about 65 mg/m². In some embodiments, paclitaxel is administered in an amount of about 70 mg/m². In some embodiments, paclitaxel is administered in an amount of about 75 mg/m². In some embodiments, paclitaxel is administered in an amount of about 80 mg/m². In some embodiments, paclitaxel is administered in an amount of about 85 mg/m². In some embodiments, paclitaxel is administered in an amount of about 90 mg/m². In some embodiments, paclitaxel is administered in an amount of about 95 mg/m². In some embodiments, paclitaxel is administered in an amount of about 100 mg/m². In some embodiments, paclitaxel is administered in an amount of about 105 mg/m². In some embodiments, paclitaxel is administered in an amount of about 110 mg/m². In some embodiments, paclitaxel is administered in an amount of about 115 mg/m². In some embodiments, paclitaxel is administered in an amount of about 120 mg/m². In some embodiments, paclitaxel is administered in an amount of about 125 mg/m². In some embodiments, paclitaxel is administered in an amount of about 130 mg/m². In some embodiments, paclitaxel is administered in an amount of about 135 mg/m². In some embodiments, paclitaxel is administered in an amount of about 140 mg/m². In some embodiments, paclitaxel is administered in an amount of about 145 mg/m². In some embodiments, paclitaxel is administered in an amount of about 150 mg/m². In some embodiments, paclitaxel is administered in an amount of about 160 mg/m². In some embodiments, paclitaxel is administered in an amount of about 170 mg/m². In some embodiments, paclitaxel is administered in an amount of about 175 mg/m². In some embodiments, paclitaxel is administered in an amount of about 180 mg/m². In some embodiments, paclitaxel is administered in an amount of about 190 mg/m². In some embodiments, paclitaxel is administered in an amount of about 200 mg/m².

In some embodiments, paclitaxel is administered in an amount of no more than 70 mg/m². In some embodiments, paclitaxel is administered in an amount of no more than 80 mg/m². In some embodiments, paclitaxel is administered in an amount of no more than 90 mg/m². In some embodiments, paclitaxel is administered in an amount of no more than 100 mg/m². In some embodiments, paclitaxel is administered in an amount of no more than 125 mg/m². In some embodiments, paclitaxel is administered in an amount of no more than 150 mg/m². In some embodiments, paclitaxel is administered in an amount of no more than 175 mg/m². In some embodiments, paclitaxel is administered in an amount of no more than 200 mg/m². In some embodiments, paclitaxel is administered in an amount of no more than 250 mg/m².

In some embodiments, the tubulin binding agent is administered in an amount of from 10 mg/m² to 150 mg/m². In some embodiments, the tubulin binding agent is administered in an amount of from 20 mg/m² to 140 mg/m². In some embodiments, the tubulin binding agent is administered in an amount of from 30 mg/m² to 130 mg/m². In some embodiments, the tubulin binding agent is administered in an amount of from 40 mg/m² to 125 mg/m². In some embodiments, the tubulin binding agent is administered in an amount of from 50 mg/m² to 110 mg/m². In some embodiments, the tubulin binding agent is administered in an amount of from 50 mg/m² to 90 mg/m². In some embodiments, the tubulin binding agent is administered in an amount of from 60 mg/m² to 100 mg/m². In some embodiments, the tubulin binding agent is administered in an amount of from 65 mg/m² to 95 mg/m². In some embodiments, the tubulin binding agent is administered in an amount of from 70 mg/m² to 90 mg/m². In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel.

In some embodiments, paclitaxel is administered in an amount of from 10 mg/m² to 150 mg/m². In some embodiments, paclitaxel is administered in an amount of from 20 mg/m² to 140 mg/m². In some embodiments, paclitaxel is administered in an amount of from 30 mg/m² to 130 mg/m². In some embodiments, paclitaxel is administered in an amount of from 40 mg/m² to 125 mg/m². In some embodiments, paclitaxel is administered in an amount of from 50 mg/m² to 110 mg/m². In some embodiments, paclitaxel is administered in an amount of from 50 mg/m² to 90 mg/m². In some embodiments, paclitaxel is administered in an amount of from 60 mg/m² to 100 mg/m². In some embodiments, paclitaxel is administered in an amount of from 65 mg/m² to 95 mg/m². In some embodiments, paclitaxel is administered in an amount of from 70 mg/m² to 90 mg/m².

In some embodiments, the tubulin binding agent is administered orally or parenterally. In some embodiments, the tubulin binding agent is administered orally. In some embodiments, the tubulin binding agent is administered parenterally. In some embodiments, the tubulin binding agent is administered intravenously. In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel.

In some embodiments, paclitaxel is administered orally or parenterally. In some embodiments, paclitaxel is administered orally. In some embodiments, paclitaxel is administered parenterally. In some embodiments, paclitaxel is administered intravenously. In some embodiments, paclitaxel mesylate is administered orally or parenterally. In some embodiments, paclitaxel mesylate is administered orally. In some embodiments, paclitaxel mesylate is administered parenterally. In some embodiments, paclitaxel mesylate is administered intravenously.

In some embodiments, the tubulin binding agent is administered over a period of less than one hour. In some embodiments, the tubulin binding agent is administered over a period of about one hour. In some embodiments, the tubulin binding agent is administered over a period of about 1.5 hours. In some embodiments, the tubulin binding agent is administered over a period of about two hours. In some embodiments, the tubulin binding agent is administered over a period of about the hours. In some embodiments, the tubulin binding agent is administered over a period of less than two hours. In some embodiments, the tubulin binding agent is administered over a period of 30-60 minutes. In some embodiments, the tubulin binding agent is administered over a period of about 45 minutes. In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel.

In some embodiments, paclitaxel is administered over a period of less than one hour. In some embodiments, paclitaxel is administered over a period of about one hour. In some embodiments, paclitaxel is administered over a period of about 1.5 hour. In some embodiments, paclitaxel is administered over a period of about two hours. In some embodiments, paclitaxel administered over a period of about the hours. In some embodiments, paclitaxel is administered over a period of less than two hours. In some embodiments, paclitaxel is administered over a period of 30-60 minutes. In some embodiments, paclitaxel is administered over a period of about 45 minutes.

In some embodiments, paclitaxel mesylate is administered over a period of less than one hour. In some embodiments, paclitaxel mesylate is administered over a period of about one hour. In some embodiments, paclitaxel mesylate is administered over a period of about 1.5 hour. In some embodiments, paclitaxel mesylate is administered over a period of about two hours. In some embodiments, paclitaxel mesylate administered over a period of about the hours. In some embodiments, paclitaxel mesylate is administered over a period of less than two hours. In some embodiments, paclitaxel mesylate is administered over a period of 30-60 minutes. In some embodiments, paclitaxel mesylate is administered over a period of about 45 minutes.

In some embodiments, the tubulin binding agent is administered no more than once a week. In some embodiments, the tubulin binding agent is administered at least once a week. In some embodiments, the tubulin binding agent is administered once a week. In some embodiments, the tubulin binding agent is administered twice a month. In some embodiments, the tubulin binding agent is administered three times a month. In some embodiments, the tubulin binding agent is administered once a month. In some embodiments, the tubulin binding agent is administered in a 28 day cycle, once a week for 3 weeks. In some embodiments, the tubulin binding agent is a taxane or a pharmaceutically acceptable salt thereof. In some embodiments, the tubulin binding agent is paclitaxel, docetaxel, cabazitaxel, or pharmaceutically acceptable salts thereof. In some embodiments, the tubulin binding agent is paclitaxel, or a pharmaceutically acceptable salt thereof. In some embodiments, the tubulin binding agent is paclitaxel. In some embodiments, the tubulin binding agent is paclitaxel mesylate.

In some embodiments, paclitaxel is administered no more than once a week. In some embodiments, paclitaxel is administered at least once a week. In some embodiments, paclitaxel is administered once a week. In some embodiments, paclitaxel is administered twice a month. In some embodiments, paclitaxel is administered three times a month. In some embodiments, paclitaxel is administered once a month. In some embodiments, paclitaxel is administered in a 28 day cycle, once a week for 3 weeks.

In some embodiments, paclitaxel mesylate is administered no more than once a week. In some embodiments, paclitaxel mesylate is administered at least once a week. In some embodiments, paclitaxel mesylate is administered once a week. In some embodiments, paclitaxel mesylate is administered twice a month. In some embodiments, paclitaxel mesylate is administered three times a month. In some embodiments, paclitaxel mesylate is administered once a month. In some embodiments, paclitaxel is administered in a 28 day cycle, once a week for 3 weeks.

### Hypersensitivity Reactions (HSRs) and Premedication

Severe, and sometimes fatal, hypersensitivity reactions (HSRs) to paclitaxel administration, including anaphylactic reactions, have been reported. It is estimated that hypersensitivity reactions occur in approximately 4% of patients, upon first exposure. (The mechanism through which paclitaxel produces hypersensitivity reactions is not well characterized, though could be due to the Cremophor EL present in traditional paclitaxel products). If no HSRs occur during initial exposure, HSRs are unlikely to occur with subsequent paclitaxel administration, and premedication is not required for subsequent paclitaxel administration. As prophylaxis for hypersensitivity reactions, a premedication regimen may be administered prior to first exposure to paclitaxel. A typical premedication regimen comprises administering:

a corticosteroid (e.g., dexamethasone, prednisone);

an antihistamine (e.g., diphenhydramine, dimethindene); and

an H₂ receptor antagonist (e.g., famotidine, ranitidine, cimetidine).

Various premedication regimens have been employed; examples include:
Oral administration of an antihistamine and a corticosteroid, 12 hours and 6 hours before infusion of paclitaxel.
Iv administration of an antihistamine, a corticosteroid, and an H₂ receptor antagonist, 30-60 minutes before infusion of paclitaxel
Dexamethasone (20 mg) over 5-10 minutes, given 30-60 minutes before infusion of paclitaxel, followed by dimethindene maleate (4 mg) and ranitidine (50 mg) over 30 minutes, all administered intravenously.
Dexamethasone (10 mg), diphenhydramine (25 mg), and cimetidine (300 mg), administered intravenously, 60 minutes before infusion of paclitaxel.
Dexamethasone (20 mg), diphenhydramine (50 mg) and famotidine (20 mg), administered intravenously, 30-60 minutes before infusion of paclitaxel.

For patients experiencing a hypersensitivity reaction, an enhanced premedication regimen may be administered, such as dexamethasone (20 mg), administered orally 12 and 6 hours prior to re-exposure with paclitaxel, in addition to diphenhydramine and cimetidine.

In some embodiments, the methods for treating diseases described herein comprise administering a combination of a tyrosine kinase inhibitor, or a pharmaceutically acceptable salt thereof and a tubulin binding agent or a pharmaceutically acceptable salt thereof, further comprise administration of a premedication. In some embodiments, the premedication comprises one or more of a corticosteroid, an antihistamine and an H₂ receptor antagonist. In some embodiments, the premedication comprises a corticosteroid, an antihistamine and an H₂ receptor antagonist. In some embodiments, the corticosteroid is dexamethasone. In some embodiments, the antihistamine is diphenhydramine. In some embodiments, the antihistamine is dimethindene maleate. In some embodiments, the H₂ receptor antagonist is famotidine. In some embodiments, the H₂ receptor antagonist is ranitidine. In some embodiments, the H₂ receptor antagonist is cimetidine. In some embodiments, the premedication is administered orally. In some embodiments, the premedication is administered intravenously. In some embodiments, one or more of the components of the premedication are administered orally and one or more of the components of the premedication are administered intravenously. In some embodiments, the premedication is administered after the administration of the tyrosine kinase inhibitor. In some embodiments, the premedication is administered before the administration of the tyrosine kinase inhibitor.

### Diseases

Described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof.

In some embodiments, the disease is a proliferative or a hyper-proliferative condition including, but not limited to cancer, hyperplasias, restenosis, inflammation, immune disorders, cardiac hypertrophy, atherosclerosis, fibrosis, pain, migraine, psoriasis, angiogenesis-related conditions or disorders, proliferation induced after medical conditions, including but not limited to surgery, angioplasty, or other conditions.

Angiogenesis-related conditions or disorders include, but are not limited to, cancers, diabetic retinopathy, proliferative retinopathy, corneal graft rejection, neovascular glaucoma, blindness and macular degeneration, erythema, psoriasis, hemophiliac joints, capillary proliferation within atherosclerotic plaques, keloids, wound granulation, vascular adhesions, rheumatoid arthritis, osteoarthritis, autoimmune diseases, Crohn's disease, restenosis, atherosclerosis, intestinal adhesions, cat scratch disease, ulcers, liver cirrhosis, glomerulonephritis, diabetic nephropathy, malignant nephrosclerosis, thrombotic microangiopathy, organ transplant rejection, glomerulopathy, diabetes, inflammation, and rodegenerative diseases.

In some embodiments, the proliferative disease is cancer. In some embodiments, the proliferative disease is non-cancerous. In some embodiments, the proliferative disease is a benign or malignant tumor. Where hereinbefore and subsequently a tumor, a tumor disease, a carcinoma or a cancer are mentioned, also metastasis in the original organ or tissue and/or in any other location are implied alternatively or in addition, regardless of the location of the tumor and/or metastasis. In some embodiments, the methods include treating, inhibiting and preventing tumor growth.

In some embodiments, the disease is cancer. In some embodiments, the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, the cancer is anal cancer, bowel cancer, colon cancer, colorectal cancer, esophageal cancer, gallbladder and biliary tract cancer, gastric cancer, gastro-intestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof.

In some embodiments, the cancer is gastric cancer. In some embodiments, the cancer is gastroesophageal junction cancer. In some embodiments, the cancer is advanced gastric cancer. In some embodiments, the cancer is advanced gastroesophageal junction cancer. In some embodiments, the cancer is recurrent gastric cancer. In some embodiments, the cancer is recurrent gastroesophageal junction cancer. In some embodiments, the cancer is metastatic gastric cancer. In some embodiments, the cancer is metastatic gastroesophageal junction cancer.

In some embodiments, the cancer comprises one or more lesions. In some embodiments, the lesion is measured before administration and either in between a plurality of administrations or after the administration or both. In some embodiments, the lesion is measured by radiological assessments using computerized tomography scan or magnetic resonance imaging. In some embodiments, the lesion has reduced in size after the administration of the combination. In some embodiments, the lesion has reduced in size by at least 10%. In some embodiments, the lesion has reduced in size by at least 20%. In some embodiments, the lesion has reduced in size by at least 25%. In some embodiments, the lesion has reduced in size by at least 30%. In some embodiments, the lesion has reduced in size by at least 40%. In some embodiments, the lesion has reduced in size by at least 50%. In some embodiments, the lesion has reduced in size by at least 60%. In some embodiments, the lesion has reduced in size by at least 70%. In some embodiments, the lesion has reduced in size by at least 75%. In some embodiments, the lesion has reduced in size by at least 80%. In some embodiments, the lesion has reduced in size by at least 90%.

Described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof. In some embodiments, the methods are a first line of therapy for treating diseases. In some embodiments, the methods are a second or a third line of therapy after the prior treatment for the disease has failed or substantially failed or the disease is substantially refractory to the first line therapy. In some embodiments, a patient has received at least one line of therapy for treating the disease prior to receiving the combination comprising a tyrosine kinase inhibitor, or a pharmaceutically acceptable salt thereof and a tubulin binding agent or a pharmaceutically acceptable salt thereof. In some embodiments, the prior line of therapy may be a line of chemotherapy or immunotherapy.

Described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof. In some embodiments, the methods further comprise administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, .beta.2-agonists, anticholinergic agents, hormonal agents, biological agents, tubulin binding agents, glucocorticoids, corticosteroid agents, antibacterial agents, antihistamines, anti-malarial agents, anti-viral agents, and antibiotics; and, optionally with radiation therapy.

Described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof. In some embodiments, the combination is administered for at least 2 months. In some embodiments, the combination is administered for about 2 months. In some embodiments, the combination is administered for about 3 months. In some embodiments, the combination is administered for about 4 months. In some embodiments, the combination is administered for about 5 months. In some embodiments, the combination is administered for about 6 months. In some embodiments, the combination is administered for about 7 months. In some embodiments, the combination is administered for about 8 months. In some embodiments, the combination is administered for about 9 months. In some embodiments, the combination is administered for about 10 months. In some embodiments, the combination is administered for about 11 months. In some embodiments, the combination is administered for about 12 months. In some embodiments, the combination is administered for more than 2 months.

### Synergy

Described herein are methods for treating diseases, where the methods comprise administering a combination of two or more therapies, in particular a combination comprising a tyrosine kinase inhibitor, or a pharmaceutically acceptable salt thereof and a tubulin binding agent or a pharmaceutically acceptable salt thereof.

In some embodiments, the combination of the tyrosine kinase inhibitor, or a pharmaceutically acceptable salt thereof and the tubulin binding agent or a pharmaceutically acceptable salt thereof, acts to produce synergistic therapeutic results.

In some embodiments, the combination of the of the tyrosine kinase inhibitor, or a pharmaceutically acceptable salt thereof and the tubulin binding agent or a pharmaceutically acceptable salt thereof, results in a joint action where one of the components supplements or enhances the action of the other component to produce an effect greater than that which may be obtained by use of the individual components in equivalent quantities, or produce effects that could not be obtained with safe quantities of the other components individually.

In some embodiments, the tyrosine kinase inhibitor, or a pharmaceutically acceptable salt thereof and the tubulin binding agent or a pharmaceutically acceptable salt thereof, work together to produce a therapeutic effect greater than the sum of their individual effects.

In some embodiments, the interaction of the tyrosine kinase inhibitor, or a pharmaceutically acceptable salt thereof and the tubulin binding agent or a pharmaceutically acceptable salt thereof is such that the addition of one compound, results in less of the other compound being required, to achieve the same therapeutic effect.

In some embodiments, administration of the tyrosine kinase inhibitor or a pharmaceutically acceptable salt thereof, results in the need for a smaller dose of the tubulin binding agent or a pharmaceutically acceptable salt thereof.

In some embodiments, administration of the tubulin binding agent or a pharmaceutically acceptable salt thereof, results in the need for a smaller dose of the tyrosine kinase inhibitor or a pharmaceutically acceptable salt thereof.

### Kits

In some embodiments, kits are provided, comprising:
a) a tyrosine kinase inhibitor, or a pharmaceutically acceptable salt thereof; and
b) a tubulin binding agent, or a pharmaceutically acceptable salt thereof;
   wherein the kits are for treating diseases.

In some embodiments, kits are provided, comprising:
a) rivoceranib, or a pharmaceutically acceptable salt thereof; and
b) a tubulin binding agent, or a pharmaceutically acceptable salt thereof;
   wherein the kits are for treating diseases.

In some embodiments, kits are provided, comprising:
a) a tyrosine kinase inhibitor, or a pharmaceutically acceptable salt thereof; and
b) paclitaxel;
   wherein the kits are for treating diseases.

In some embodiments, kits are provided, comprising:
a) a tyrosine kinase inhibitor, or a pharmaceutically acceptable salt thereof; and
b) a tubulin binding agent, or a pharmaceutically acceptable salt thereof;
   wherein the kits are for treating cancer.

In some embodiments, kits are provided, comprising:
a) rivoceranib, or a pharmaceutically acceptable salt thereof; and
b) a tubulin binding agent, or a pharmaceutically acceptable salt thereof;
   wherein the kits are for treating cancer.

In some embodiments, kits are provided, comprising:
a) a tyrosine kinase inhibitor, or a pharmaceutically acceptable salt thereof; and
b) paclitaxel;
   wherein the kits are for treating cancer.

In some embodiments, kits are provided, comprising:
a) a tyrosine kinase inhibitor, or a pharmaceutically acceptable salt thereof; for once daily oral administration; and
b) a tubulin binding agent, or a pharmaceutically acceptable salt thereof, for once weekly iv administration;
   wherein the kits are for treating diseases.

In some embodiments, kits are provided, comprising a tyrosine kinase inhibitor, or a pharmaceutically acceptable salt thereof, present in an amount effective to enhance the efficacy of the tubulin binding agent to treat diseases.

In some embodiments, kits are provided, comprising a tubulin binding agent, or a pharmaceutically acceptable salt thereof, present in an amount effective to enhance the efficacy of the tyrosine kinase inhibitor to treat diseases.

In some embodiments, kits are provided, comprising:
a) a tyrosine kinase inhibitor, or a pharmaceutically acceptable salt thereof;
b) a tubulin binding agent, or a pharmaceutically acceptable salt thereof;
c) a corticosteroid;
d) an antihistamine; and
e) an H₂ receptor antagonist;
   wherein the kits are for treating diseases

In some embodiments, packaged pharmaceutical therapies are provided, comprising:
a) a tyrosine kinase inhibitor, or a pharmaceutically acceptable salt thereof;
b) a tubulin binding agent, or a pharmaceutically acceptable salt thereof;
c) a container; and
d) instructions for use of the therapy to treat a disease or condition in a mammal.

A kit for treating cancer, comprising
i) rivoceranib, or a pharmaceutically acceptable salt thereof, for once daily oral administration; and
ii) paclitaxel for once weekly iv administration; and
iii) one or more of:
   a) a corticosteroid;
   b) an antihistamine; and
   c) an H₂ receptor antagonist.

### EXAMPLES

These examples are provided for illustrative purposes only and not to limit the scope of the claims provided herein.

### Example 1: An Open-Label, Single-Arm, Dose Escalation and Dose Expansion Phase I/IIa Study to Determine the Maximum Tolerated Dose (MTD) and Recommended Phase 2 Dose (RP2D), as a Second-Line Therapy in Advanced, Recurrent and/or Metastatic Gastric or Gastroesophageal Junction Cancer

Objectives: Phase I objectives are to determine the maximum tolerated dose (MID) and recommended. Phase 2 dose (RP2D). Phase II objectives are to determine clinical activity, efficacy, safety, tolerability and measure PK parameters. Efficacy is evaluated by radiological assessments of target lesions using computerized tomography scan or magnetic resonance imaging, performed approximately every 8 weeks.

Patients: Up to 38 patients are enrolled into the study.

The study population includes patients with metastatic gastric cancer and gastroesophageal cancer who have progressed first-line of platinum and/or fluoropyrimidine therapy, and have at least one measurable lesion.

Phase I patients are enrolled in Cycle 1 of three successive dose level cohorts, to determine the maximum tolerated dose and recommended Phase 2 dose. Phase IIa patients are enrolled in Cycle 1 at the recommended Phase 2 dose, (as determined in phase I). All patients receive rivoceranib in combination with paclitaxel in 4-week cycles (28 days). All patients receive premedication administered by intravenous infusion (dexamethasone 20 mg, diphenhydramine 50 mg and famotidine 20 mg) before paclitaxel is administered.

Dose Determination: Rivoceranib 685 mg has been studied as a single agent in patients with advanced gastric cancer in Phase 2 and Phase 3 studies. In these studies, once daily rivoceranib 685 mg demonstrated efficacy against gastric cancer with manageable toxicity, establishing once daily rivoceranib 685 mg as the recommended dose for gastric cancer. A Phase 3 study is ongoing, using a starting dose of 700 mg rivoceranib. In the current study, paclitaxel (at the standard dosing of 80 mg/m², weekly) forms the primary treatment. The lower starting dose of 400 mg rivoceranib and has proven a safe dose in multiple previous studies. The combined use of rivoceranib and paclitaxel enhances the antitumor activity of both agents for improved progression-free and overall survival.

Dose escalation (Phase I): The starting dose is continuous daily 400 mg oral rivoceranib in combination with intravenous (IV) paclitaxel 80 mg/m² administered on Days 1, 8, and 15 of the 28-day cycle. Escalation is to 500 mg and 600 mg; de-escalation level is to 300 mg.

At least 3 patients are enrolled into each dose level with a minimum interval of 7 days between the first patient and the next 2 patients of each dose level.

The study follows a 3+3 dose-escalation scheme until maximum tolerated dose is established; dose escalation, dose de-escalation, or dose level expansion proceeds based on the occurrence of DLTs at each dose level.

Evaluation of a cohort of at least 3 patients completing Cycle 1 at that dose level is required prior to determining the next dose level for the next cohort.

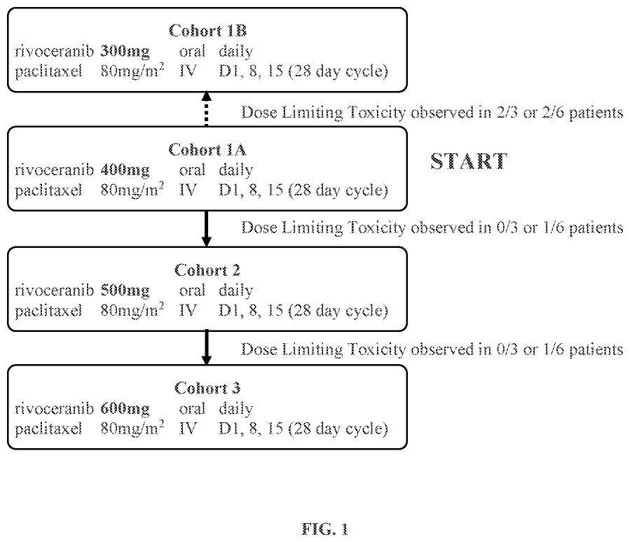

If a DLT is observed in 1 of 3 patients, 3 additional patients are enrolled at the same dose.
If a DLT is observed in 1 of the 6 patients —no additional patients are required
the next dose level is opened.
   If a DLT is observed in ≥2 patients —the previous dose level is the MTD
   After completion of Cycle 1 patients continue therapy.
   The study scheme is shown in FIG. 1.

Study Treatment Details: Rivocernib is provided as 100 mg and 200 mg film-coated tablets. The tablet composition includes rivocernib, pregelatinized starch, microcrystalline cellulose, sodium starch glycolate, povidone (K-29/32), colloidal silicon dioxide, magnesium stearate, Opadry white, and water. Rivoceranib doses for the study are 400 mg, 500 mg, 600 mg, and optionally 300 mg administered orally, once daily, and administration times should be consistent throughout the study period. Paclitaxel is administered intravenously over a 60 minute period, after dosing of rivoceranib.

### Example 2: Multiple Drug Related Transporter Inhibition Assay (P-gp and BCRP)

Objective: To assess the potential of rivoceranib to inhibit the transporter-mediated basolateral to apical (B-A apparent permeability (Pₐₚₚ) of a radiolabelled P-gp substrate ([³H]-digoxin) across MDCK-MDR1 cells and BCRP probe ([³H]-estrone 3-sulfate) across Caco-2 cells.

### Experimental Procedures:

P-gp inhibition assay: MDCK-MDR1 cells obtained from the NIH (Rockville, Md., USA) are used between passage numbers 6-30. Cells are seeded onto Millipore Multiscreen Transwell plates at 3.4×10⁵ cells/cm². The cells are cultured in DMEM and media is changed on day 3. On day 4 the P-gp inhibition study is performed. Cell culture and assay incubations are carried out at 37° C. in an atmosphere of 5% CO₂ with a relative humidity of 95%. On the day of the assay, the monolayers are prepared by rinsing both basolateral and apical surfaces twice with warmed (37° C.) transport buffer (HBSS containing 25 mM HEPES and 4.45 mM glucose, pH 7.4). Cells are then pre-incubated with transport buffer containing test compound or positive control inhibitor (cyclosporin A) in both apical and basolateral compartments for 30 min at 37° C. Dosing solutions are prepared by diluting digoxin, and test compound where applicable, in transport buffer to give a final digoxin concentration of 5 μM (final DMSO concentration 1% v/v). The fluorescent integrity marker lucifer yellow is prepared in receiver solutions in vehicle or test compound-containing transport buffer. After pre-incubation, the transport buffer is removed from both apical and basolateral compartments and replaced with the appropriate dosing or receiver solution. For assessment of B-A permeability, transport buffer is removed from the basolateral companion plate and replaced with dosing solution. Fresh transport buffer containing lucifer yellow and test compound where applicable (final DMSO concentration 1% v/v) is added to the apical compartment insert, which is then placed into the companion plate.

After a 90 min incubation the apical compartment inserts and the companion plates are separated and compartments sampled for analysis. Seven concentrations of test compound (typically 0.3, 1, 3, 10, 30, 100 and 300 μM for orally administered drugs; reflecting high intestinal luminal concentrations) are assessed in addition to a vehicle control (0 μM). However, the choice of concentration range is dependent on test compound solubility and the range may be increased or decreased accordingly. A triplicate determination of each test compound concentration is performed. The positive control inhibitor is evaluated in parallel. [³H]-Digoxin is quantified by liquid scintillation counting to give disintegrations per minute (dpm). The integrity of the monolayers throughout the experiment is checked by monitoring lucifer yellow permeation using fluorimetric analysis. Lucifer yellow permeation is high if monolayers have been damaged. If a lucifer yellow Pₐₚₚ value is above a pre-defined threshold in one well, but the derived Pₐₚₚ result for probe substrate in that well is qualitatively similar to that determined in the remaining replicate wells (within the lucifer yellow threshold) then, based upon the scientific judgement of the responsible scientist, the cell monolayer may be considered acceptable. If this is not the case, then the result from the affected monolayer is excluded from the IC₅₀ determination. The impact of any exclusion will be assessed and a decision will be made on whether to repeat the assay based on the reliance of the data point on the accuracy of the IC₅₀.

BCRP inhibition assay: Caco-2 cells obtained from the ATCC are used between passage numbers 40-60. Cells are seeded onto Millipore Multiscreen Transwell plates at 1×10⁵ cells/cm². The cells are cultured in DMEM and media is changed every two or three days. On day 20 the BCRP inhibition study is performed. Cell culture and assay incubations are carried out at 37° C. in an atmosphere of 5% CO₂ with a relative humidity of 95%. On the day of the assay, the monolayers are prepared by rinsing both apical and basolateral surfaces twice with warmed (37° C.) transport buffer (HBSS containing 25 mM HEPES and 4.45 mM glucose, pH 7.4). Cells are then pre-incubated with transport buffer containing test compound or positive control inhibitor (novobiocin) in both apical and basolateral compartments for 30 min at 37° C. Dosing solutions are prepared by diluting estrone 3-sulfate, and test compound where applicable, in transport buffer to give a final estrone 3-sulfate concentration of 1 μM (final DMSO concentration 1% v/v). The fluorescent membrane integrity marker lucifer yellow is prepared in receiver solutions in vehicle or test compound-containing transport buffer. After pre-incubation, the transport buffer is removed from both apical and basolateral compartments and replaced with the appropriate dosing or receiver solution. For assessment of B-A permeability, transport buffer is removed from the basolateral companion plate and replaced with dosing solution. Fresh transport buffer containing lucifer yellow and test compound where applicable (final DMSO concentration 1% v/v) is added to the apical compartment insert, which is then placed into the companion plate. After a 90 min incubation the apical compartment inserts and the companion plates are separated and compartments sampled for analysis. Seven concentrations of test compound (typically 0.3, 1, 3, 10, 30, 100 and 300 μM for orally administered drugs; reflecting high intestinal luminal concentrations) are assessed in addition to a vehicle control (0 μM). However, the choice of concentration range is dependent on test compound solubility and the range may be increased or decreased accordingly. A triplicate determination of each test compound concentration is performed. The positive control inhibitor is evaluated in parallel. [³H]-Estrone 3-sulfate is quantified by liquid scintillation counting to give disintegrations per minute (dpm). The integrity of the monolayers throughout the experiment is checked by monitoring lucifer yellow permeation using fluorimetric analysis. Lucifer yellow permeation is high if monolayers have been damaged. If a lucifer yellow Pₐₚₚ value is above a pre-defined threshold in one well, but the derived Pₐₚₚ result for probe substrate in that well is qualitatively similar to that determined in the remaining replicate wells (within the lucifer yellow threshold) then, based upon the scientific judgement of the responsible scientist, the cell monolayer may be considered acceptable. If this is not the case, then the result from the affected monolayer is excluded from the IC₅₀ determination. The impact of any exclusion will be assessed and a decision will be made on whether to repeat the assay based on the reliance of the data point on the accuracy of the IC₅₀.

Data Analysis: Corrected B-A Pₐₚₚ of probe substrate is calculated by subtracting its mean passive Pₐₚₚ determined in the presence of the highest concentration of positive control inhibitor (giving 100% transporter inhibition). The mean corrected B-A Pₐₚₚ from vehicle wells (0 μM test compound) is defined as 100% transport activity and this value is then used to calculate the percentage control transport activity for all other test compound concentrations. Percentage control transport activity is plotted against test compound concentration and fitted to calculate an IC₅₀ value (test compound concentration which produces 50% inhibition of transport activity).

Results: IC₅₀ against P-gp and BCRP was 15.6±2.7 μM and 0.7±0.06 respectively. These results indicate that rivoceranib has potential to enhance the anti-tumor activities of concomitant taxane treatment through inhibiting multiple drug resistant-related transporters.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating cancer, comprising administering:
a) rivoceranib, or a pharmaceutically acceptable salt thereof; and
b) paclitaxel, or a pharmaceutically acceptable salt thereof.

2. The method of claim 1, comprising administering rivoceranib.

3. The method of claim 1, comprising administering a pharmaceutically acceptable salt of rivoceranib.

4. The method of claim 3, wherein the pharmaceutically acceptable salt of rivoceranib is a mesylate salt.

5. The method of claim 1, wherein the rivoceranib is administered in an amount of from 100 mg to 1000 mg.

6. The method of claim 1, wherein the rivoceranib is administered in an amount of from 150 mg to 800 mg.

7. The method of claim 1, wherein the rivoceranib is administered in an amount of from 200 mg to 700 mg.

8. The method of claim 1, wherein the rivoceranib is administered in an amount of less than 700 mg.

9. The method of claim 1, wherein the rivoceranib is administered in an amount of about 200 mg.

10. The method of claim 1, wherein the rivoceranib is administered in an amount of about 300 mg.

11. The method of claim 1, wherein the rivoceranib is administered in an amount of about 400 mg.

12. The method of claim 1, wherein the rivoceranib is administered in an amount of about 500 mg.

13. The method of claim 1, wherein the rivoceranib is administered in an amount of about 600 mg.

14. The method of claim 1, wherein the total daily dose of the rivoceranib is less than 700 mg.

15. The method of claim 1, wherein the total daily dose of the rivoceranib is less than 685 mg.

16. The method of claim 1, wherein the rivoceranib is administered orally.

17. The method of claim 1, wherein the rivoceranib is administered as a dried powder, a liquid, a capsule, a pellet or a tablet.

18. The method of claim 1, wherein the rivoceranib is administered as a tablet.

19. The method of claim 18, wherein the tablet is a film coated tablet

20. The method of claim 18, wherein the tablet comprises the rivoceranib in an amount of about 100 mg.

21. The method of claim 18, wherein the tablet comprises the rivoceranib in an amount of about 200 mg.

22. The method of claim 18, wherein the tablet further comprises one or more of pregelatinized starch, microcrystalline cellulose, sodium starch glycolate, povidone (K-29/32), colloidal silicon dioxide, magnesium stearate and Opadry white.

23. The method of claim 1, wherein the rivoceranib is administered once daily.

24. The method of claim 1, wherein the rivoceranib is administered twice daily.

25. The method of claim 1, comprising administering paclitaxel.

26. The method of claim 1, comprising administering a pharmaceutically acceptable salt of paclitaxel.

27. The method of claim 1, comprising administering paclitaxel in an amount of no more than 100 mg/m².

28. The method of claim 1, wherein the paclitaxel is administered in an amount of 10 mg/m² to 100 mg/m².

29. The method of claim 1, wherein the paclitaxel is administered in an amount of 50 mg/m² to 90 mg/m².

30. The method of claim 1, wherein the paclitaxel is administered in an amount of about 80 mg/m².

31. The method of claim 1, wherein the paclitaxel is administered orally or parenterally.

32. The method of claim 1, wherein the paclitaxel is administered parenterally.

33. The method of claim 32, wherein the parenteral administration is selected from intravenous, intradermal, intramuscular or subcutaneous administration.

34. The method of claim 1, wherein the paclitaxel is administered intravenously.

35. The method of claim 1, wherein the paclitaxel is administered after the administration of the rivoceranib.

36. The method of claim 1, wherein the paclitaxel is administered about an hour after the administration of the rivoceranib.

37. The method of claim 1, wherein the paclitaxel is administered over a period of less than one hour.

38. The method of claim 1, wherein the paclitaxel is administered over a period of about an hour.

39. The method of claim 1, wherein the paclitaxel is administered over a period of 30-60 minutes.

40. The method of claim 1, wherein the paclitaxel is administered no more than once a week.

41. The method of claim 1, wherein the paclitaxel is administered at least once a week.

42. The method of claim 1, wherein the paclitaxel is administered once a week.

43. The method of claim 1, wherein the paclitaxel is administered once a month.

44. The method of claim 1, wherein the paclitaxel is administered twice a month.

45. The method of claim 1, wherein the paclitaxel is administered three times a month.

46. The method of claim 1, wherein the paclitaxel is administered as a composition comprising nanoparticles comprising paclitaxel and a protein carrier.

47. The method of claim 1, wherein the paclitaxel is administered as a composition comprising nanoparticles comprising paclitaxel and albumin.

48. The method of claim 47, wherein the paclitaxel in the nanoparticles is coated with the albumin.

49. The method of claim 47, wherein the nanoparticles have an average diameter of no greater than about 200 nm.

50. The method of claim 47, wherein the weight ratio of albumin and paclitaxel in the nanoparticle composition is about 9:1.

51. The method of claim 1, wherein prior to the administration of the paclitaxel, premedication is administered.

52. The method of claim 51, wherein the premedication comprises one or more of dexamethasone, diphenhydramine or famotidine.

53. The method of claim 51, wherein the premedication comprises dexamethasone, diphenhydramine and famotidine.

54. The method of claim 51, wherein the premedication comprises:
20 mg dexamethasone;
50 mg diphenhydramine; and
20 mg famotidine.

55. The method of claim 51, wherein the premedication is administered intravenously.

56. The method of claim 1, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

57. The method of claim 1, wherein the cancer is a cancer that affects the digestive system.

58. The method of claim 1, wherein the cancer is anal cancer, bowel cancer, colon cancer, colorectal cancer, esophageal cancer, gallbladder and biliary tract cancer, gastric cancer, gastro-intestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof.

59. The method of claim 1, wherein the cancer is gastric cancer.

60. The method of claim 1, wherein the cancer is gastroesophageal junction cancer.

61. The method of claim 1, wherein the cancer is advanced gastric or gastroesophageal junction cancer.

62. The method of claim 1, wherein the cancer is recurrent metastatic gastric or gastroesophageal junction cancer.

63. The method of claim 1, wherein the cancer is metastatic gastric or gastroesophageal junction cancer.

64. The method of claim 1, wherein the method is a second line therapy.

65. The method of claim 1, further comprising administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, β2-agonists, anticholinergic agents, hormonal agents, biological agents, tubulin binding agents, glucocorticoids, corticosteroid agents, antibacterial agents, antihistamines, antiemetic agents, anti-malarial agents, anti-viral agents, and antibiotics; and, optionally with radiation therapy.

66. The method of claim 1, further comprising administering radiation therapy.

67. The method of claim 1, wherein the cancer comprises a lesion.

68. The method of claim 67, wherein the lesion is measured before administration and either in between a plurality of administrations or after the administration or both.

69. The method of claim 67, wherein the lesion is measured by radiological assessments using computerized tomography scan or magnetic resonance imaging.

70. The method of claim 67, wherein the lesion has reduced in size after the administration of the combination.

71. The method of claim 70, wherein the lesion has reduced in size by at least 10%.

72. The method of claim 70, wherein the lesion has reduced in size by at least 20%.

73. The method of claim 70, wherein the lesion has reduced in size by at least 50%.

74. The method of claim 70, wherein the lesion has reduced in size by at least 75%.

75. A method for treating cancer, comprising administering a combination of
a) a tyrosine kinase inhibitor, or a pharmaceutically acceptable salt thereof; and
b) a tubulin binding agent, or a pharmaceutically acceptable salt thereof.

76. The method of claim 75, wherein the tyrosine kinase inhibitor is a vascular endothelial growth factor receptor (VEGF) inhibitor.

77. The method of claim 75, wherein the tyrosine kinase inhibitor is a selective vascular endothelial growth factor receptor-2 (VEGF2) inhibitor.

78. The method of claim 75, wherein the tyrosine kinase inhibitor is afatinib, alectinib, apatinib, axitinib, bosutinib, brigatinib, canertinib, crizotinib, ceritinib, dasatinib, danusertib, dabrafenib, erlotinib, gefitinib, ibrutinib, imatinib, lapatinib, lenvatinib, neratinib, nilotinib, nintedanib, osimertinib, palbociclib, pazopanib, pegaptanib, ponatinib, ranibizumab, rebastinib, regorafenib, ribociclib, rivoceranib, ruxolitinib, semaxinib, sorafenib, sunitinib, tivozanib, trametinib, tofacitinib, vandetanib, vatalanib, vemurafenib or vismodegib.

79. The method of claim 75, wherein the tyrosine kinase inhibitor is rivoceranib.

80. The method of claim 75, wherein the tyrosine kinase inhibitor is rivoceranib mesylate.

81. The method of claim 75, wherein the tyrosine kinase inhibitor is administered in an amount of from 150 mg to 800 mg.

82. The method of claim 75, wherein the tyrosine kinase inhibitor is administered in an amount of from 200 mg to 700 mg.

83. The method of claim 75, wherein the tyrosine kinase inhibitor is administered in an amount of less than 700 mg.

84. The method of claim 75, wherein the tyrosine kinase inhibitor is administered in an amount of about 200 mg.

85. The method of claim 75, wherein the tyrosine kinase inhibitor is administered in an amount of about 300 mg.

86. The method of claim 75, wherein the tyrosine kinase inhibitor is administered in an amount of about 400 mg.

87. The method of claim 75, wherein the tyrosine kinase inhibitor is administered in an amount of about 500 mg.

88. The method of claim 75, wherein the tyrosine kinase inhibitor is administered in an amount of about 600 mg.

89. The method of claim 75, wherein the total daily dose of the tyrosine kinase inhibitor is less than 700 mg.

90. The method of claim 75, wherein the total daily dose of the tyrosine kinase inhibitor is less than 685 mg.

91. The method of claim 75, wherein the tyrosine kinase inhibitor is administered orally.

92. The method of claim 75, wherein the tyrosine kinase inhibitor is administered as a tablet.

93. The method of claim 75, wherein the tyrosine kinase inhibitor is administered once daily.

94. The method of claim 75, wherein the tyrosine kinase inhibitor is administered twice daily.

95. The method of claim 75, wherein the tubulin binding agent is a taxane.

96. The method of claim 95, wherein the taxane is paclitaxel, Docetaxel, Cabazitaxel, Larotaxel, Orataxel, Tesetaxel, Milataxel, Taxoprexin, docetaxel-d₆-t-Boc, docetaxel-f3-t-Boc, cabazitaxel-7,10-d₆, abeo-taxane15a.2, BMS-184476, BMS-188797, BMS-275183, SB-T-1214, SB-T-1216, SB-T-12854, SB-T-121602, SB-CST-10202 or DHA-SB-T-1214.

97. The method of claim 75, wherein the tubulin binding agent is administered in an amount of no more than 100 mg/m².

98. The method of claim 75, wherein the tubulin binding agent is administered in an amount of 10 mg/m² to 100 mg/m².

99. The method of claim 75, wherein the tubulin binding agent is administered in an amount of 50 mg/m² to 90 mg/m².

100. The method of claim 75, wherein the tubulin binding agent is administered in an amount of about 80 mg/m².

101. The method of claim 75, wherein the tubulin binding agent is administered orally or parenterally.

102. The method of claim 75, wherein the tubulin binding agent is administered parenterally.

103. The method of claim 75, wherein the tubulin binding agent is administered intravenously.

104. The method of claim 75, wherein the tubulin binding agent is administered after the administration of the tyrosine kinase inhibitor.

105. The method of claim 75, wherein the tubulin binding agent is administered about an hour after the administration of the tyrosine kinase inhibitor.

106. The method of claim 75, wherein the tubulin binding agent is administered over a period of less than one hour.

107. The method of claim 75, wherein the tubulin binding agent is administered over a period of about an hour.

108. The method of claim 75, wherein the tubulin binding agent is administered over a period of 30-60 minutes.

109. The method of claim 75, wherein the tubulin binding agent is administered no more than once a week.

110. The method of claim 75, wherein the tubulin binding agent is administered at least once a week.

111. The method of claim 75, wherein the tubulin binding agent is administered once a week.

112. The method of claim 75, wherein the tubulin binding agent is administered once a month.

113. The method of claim 75, wherein the tubulin binding agent is administered twice a month.

114. The method of claim 75, wherein prior to the administration of the tubulin binding agent, premedication is administered.

115. The method of claim 114, wherein the premedication comprises one or more of dexamethasone, diphenhydramine or famotidine.

116. The method of claim 114, wherein the premedication comprises dexamethasone, diphenhydramine and famotidine.

117. The method of claim 114, wherein the premedication comprises:
20 mg dexamethasone;
50 mg diphenhydramine; and
20 mg famotidine.

118. The method of claim 114, wherein the premedication is administered intravenously.

119. The method of claim 75, wherein the tubulin binding agent is administered.

120. The method of claim 75, wherein the rivoceranib and the paclitaxel act synergistically to treat the cancer.

121. The method of claim 75, wherein the administration of the rivoceranib and the paclitaxel is more effective than administering the rivoceranib alone or paclitaxel alone.

122. The method of claim 75, wherein the cancer is selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

123. The method of claim 75, wherein the cancer is a cancer that affects the digestive system.

124. The method of claim 75, wherein the cancer is anal cancer, bowel cancer, colon cancer, colorectal cancer, esophageal cancer, gallbladder and biliary tract cancer, gastric cancer, gastro-intestinal stromal tumor (gist), gastroesophageal junction cancer, intestinal cancer, liver cancer, neuroendocrine tumors, pancreatic cancer, peritoneal cancer, rectal cancer, small bowel cancer, stomach cancer, or a combination thereof.

125. The method of claim 75, wherein the cancer is gastric cancer.

126. The method of claim 75, wherein the cancer is gastroesophageal junction cancer.

127. The method of claim 75, wherein the cancer is advanced gastric or gastroesophageal junction cancer.

128. The method of claim 75, wherein the cancer is recurrent metastatic gastric or gastroesophageal junction cancer.

129. The method of claim 75, wherein the cancer is metastatic gastric or gastroesophageal junction cancer.

130. The method of claim 75, wherein the method is a second line therapy.

131. The method of claim 75, further comprising administering one or more additional agents selected from the group consisting of anti-cancer agents, anti-proliferative agents, chemotherapeutic agents, immunomodulatory agents, anti-angiogenic agents, anti-inflammatory agents, alkylating agents, steroidal and non-steroidal anti-inflammatory agents, pain relievers, leukotriene antagonists, .beta.2-agonists, anticholinergic agents, hormonal agents, biological agents, tubulin binding agents, glucocorticoids, corticosteroid agents, antibacterial agents, antihistamines, anti-malarial agents, anti-viral agents, and antibiotics; and, optionally with radiation therapy.

132. The method of claim 75, wherein the cancer comprises a lesion.

133. The method of claim 132, wherein the lesion is measured before administering the combination and either in between a plurality of administrations of the combination or after the administration of the combination or both.

134. The method of claim 133, wherein the lesion is measured by radiological assessments using computerized tomography scan or magnetic resonance imaging.

135. The method of claim 132, wherein the lesion has reduced in size after the administration of the combination.

136. The method of claim 135, wherein the lesion has reduced in size by at least 10%.

137. The method of claim 135, wherein the lesion has reduced in size by at least 20%.

138. The method of claim 135, wherein the lesion has reduced in size by at least 50%.

139. The method of claim 135, wherein the lesion has reduced in size by at least 75%.

140. The method of claim 135, wherein the lesion has reduced in size by more than if rivoceranib alone or paclitaxel alone had been administered.

141. A method for treating cancer, comprising administering a combination of
a) rivoceranib, or a pharmaceutically acceptable salt thereof; and
b) no more than 80 mg/m² paclitaxel.

142. A method for treating cancer, comprising administering a combination of
a) no more than 685 mg rivoceranib, or a pharmaceutically acceptable salt thereof; and
b) paclitaxel.

143. A method for treating cancer, comprising administering a combination of
a) no more than 685 mg rivoceranib, or a pharmaceutically acceptable salt thereof; and
b) no more than 80 mg/m² paclitaxel.

144. A method for treating cancer, comprising administering a combination of
a) rivoceranib, or a pharmaceutically acceptable salt thereof; and
b) paclitaxel;
wherein the rivoceranib and the paclitaxel act synergistically.
